(19) United States
(12) Patent Application Publication
(10) Pub. No.: US 2002/0081711 A1
(43) Pub. Date: Jun. 27, 2002

(54) Novel genetic suppressor elements and methods of making the same

(75) Inventors: Meenhard Herlyn, Wynnewood, PA (US); Kapaettu Satyamoorthy, Karnataka (IN)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: The Wistar Institute, Philadelphia, PA

(21) Appl. No.: 09/967,432

(22) Filed: Sep. 28, 2001

Related U.S. Application Data
(63) Continuation of application No. PCT/US00/07807, filed on Mar. 24, 2000.
(60) Provisional application No. 60/126,479, filed on Mar. 26, 1999.

(51) Int. Cl. A61K 48/00; C12N 7/01; C12N 15/867

(52) U.S. Cl. 435/235.1; 435/456; 435/320.1; 424/93.21

(57) ABSTRACT

The invention includes compositions for inhibiting a phenotype associated with diseased cells in a mammal and compositions and methods for generating genetic suppressor elements for inhibiting such phenotypes. The invention further includes pharmaceutical compositions and methods of treatment for mammals (e.g. humans) afflicted with melanoma or other solid tumors.

### CROSS-REFERENCE TO RELATED APPLICATIONS

This application is a continuation of international patent application PCT/US00/07807, filed on Mar. 24, 2000. This application is also entitled to priority pursuant to 35 U.S.C. §119(e) to U.S. provisional patent application No. 60/126,479, which was filed on Mar. 26, 1999.

### STATEMENT REGARDING FEDERALLY SPONSORED RESEARCH OR DEVELOPMENT

This research was supported in part by U.S. Government funds (National Institutes of Health grant number CA47159), and the U.S. Government may therefore have certain rights in the invention.

### BACKGROUND OF THE INVENTION

Genetic suppressor elements (GSEs) are short fragments of genes which are capable of inhibiting the function of the gene from which they are derived (Roninson et al., 1995, Cancer Res. 55:4023-4028; Holzmayer et al., 1992, Nucl. Acid Res. 20:711-717). Antisense-oriented GSEs may encode efficient inhibitory antisense RNA molecules and sense-oriented GSEs may affect translation and/or stability of RNA from which they are derived and/or domains of proteins that interfere with the protein in a dominant negative fashion. This strategy has been successfully used to isolate GSEs for topoisomerase II, p53, etoposide resistance, apoptosis resistance, and growth inhibition (Gudkov et al., 1993, Proc. Natl. Acad. Sci USA 90:3231-3235; Ossovskaya et al., 1996, Proc. Natl. Acad. Sci. USA 93:10309-10314; Gallagher et al., 1997, Oncogene 14:185-193; Gudkov et al., 1994, Proc. Natl. Acad. Sci. USA 91:3744-3748; Deiss et al., 1991, Science 252:117-120; Kissil et al., 1995, J. Biol. Chem. 270:27932-27936; Vito et al., 1996, Science 271:521-525; Garkavtsev et al., 1996, Nature Genet. 14:415-420).

Adhesion molecules are an integral part of the tumor cell surface and also play a crucial role in tumor growth, migration and invasion. An example of such an adhesion molecule is Mel-CAM, also referred to as MUC18, MCAM, S-endo-1 antigen, or CD146 which is a cell surface glycoprotein having five immunoglobulin-like domains, and which mediates adhesion between melanoma cells (Lehmann et al., 1989, Proc. Natl. Acad. Sci. USA 86:9891-9895; Shih et al., 1994, Cancer Res. 54:2514-2520; Shih et al., 1997, Cancer Res. 57:3835-3840; Sers et al., 1993, Proc. Natl. Acad. Sci. USA 90:8514-8518). Mel-CAM expression levels in melanomas are associated with increasing tumor thickness, poor survival and metastasis formation (Xie et al., 1997, Cancer Res. 57:2295-2303; Kraus et al., 1997, Melanoma Res. 7(Suppl. 2):S75-S81; Wang et al., 1996, Cell Growth Differ. 7:1733-1740). Indeed, all metastatic melanomas express Mel-CAM (Shih et al., 1998, Modem Pathol. 11:1098-1106). Transduction of non-tumorigenic, radial growth phase 3-like (RGP³) melanoma cells with Mel-CAM cDNA confers tumorigenicity and increased invasiveness to these cells (Sers et al., 1993, Proc. Natl. Acad. Sci. USA 90:8514-8518; Xie et al., 1997, Cancer Res. 57:2295-2303). Mel-CAM has been shown to bind an unidentified heterophilic ligand found on melanoma cells (Shih et al., 1997, Cancer Res. 57:3835-3840; Johnson et al., 1997, Int. J. Cancer 73:769-774).

In facilitating metastasis, Mel-CAM is activated by cAMP (Xie et al., 1997, Cancer Res. 57:2295-2303; Rummel et al., 1996, Cancer Res. 56:2218-2223), modulated by the transcription factor AP-2 (Jean et al., 1998, J. Biol. Chem. 273:16501-16508), and regulated in cultured melanocytes by direct contact with keratinocytes (Shih et al., 1994, Am. J. Pathol. 145:837-845).

Melanomas and other types of cancers remain significant public health menaces. There is a significant need in the art for the development of efficacious methods of reducing cancerous and other disease-related phenotypes. The present invention satisfies this need.

### BRIEF SUMMARY OF THE INVENTION

The invention relates to a trans-recoverable packaging-deficient retrovirus vector. The vector comprises a retrovirus having a genome which comprises a portion derived from the sequence of a cDNA corresponding to a protein expressed in a diseased cell. The portion has a length of less than about 3,000 nucleotide residues. The vector lacks a functional copy of a gene necessary for packaging of progeny of the vector. The portion may be complementary to or homologous with the cDNA. In one embodiment, the cDNA corresponds to a cell surface adhesion protein of a diseased cell such as a melanoma cell. For example, the protein may be Mel-CAM or integrin (beta)3.

The vector may, for example, lack a functional copy of a gene selected from the group consisting of the gag gene, the pol gene, and the env gene of the retrovirus. The vector may be derived from a retrovirus selected from the group consisting of a Molony murine leukemia virus and a Molony murine sarcoma virus. For example, it may be a PG1EN vector comprising the portion.

In certain embodiments of the vector of the invention, the vector further comprises a selectable marker. The portion may be operably linked with a promoter/enhancer region, with an ATG codon, with a stop codon, or with some combination of these. Furthermore, the portion may be operably linked with a selectable marker and an internal ribosome entry site interposed therebetween.

In multiple embodiments, the invention includes a pharmaceutical composition comprising a vector and as pharmaceutically acceptable carrier, and optionally, the invention includes a pharmaceutical composition comprising a genetic suppressor element and as pharmaceutically acceptable carrier.

The invention also relates to a library comprising a plurality of the vector of the invention. At least two of the vectors collectively comprise different portions derived from the sequence of the same cDNA. Preferably, the vectors collectively comprise at least 10 different portions derived from the sequence of the cDNA. The portions may, for example, be generated by random cleavage of the cDNA or by amplification of sequential regions of the cDNA. In one embodiment, the cDNA corresponds to Mel-CAM and the portions are derived from at least one region selected from the group consisting of SEQ ID NOs: 1-9. In another embodiment, the cDNA corresponds to integrin (beta)3 and the portions are derived from at least one region selected from the group consisting of SEQ ID NOs: 10-21.

The invention further relates to a method of generating a genetic suppressor element which suppresses an undesirable phenotype in a diseased cell. This method comprises

a) contacting a retrovirus library with a population of target cells, and

b) performing at least one selection cycle using the population. The selection cycle comprises selecting a fraction of the target cells which express the selectable marker and which exhibit suppression of the undesirable phenotype. The library comprises a plurality of individual retrovirus particles. Individual retrovirus particles comprise a selectable marker and a fragment of an RNA which is transcribed in the diseased cell. The fragment has a length less than about 3,000 nucleotide residues and is operably linked with an ATG codon. The retrovirus particles lack a component necessary for packaging of progeny retrovirus particles. The target cells are susceptible to infection by the retrovirus particles. Preferably, at least two selection cycles are performed and cells of the fraction are propagated between the selection cycles.

In one embodiment, this method further comprises

c) providing the component to cells of the fraction. Progeny retrovirus particles comprising the genetic suppressor element are thereby generated.

The method may further comprise

d) isolating the genetic suppressor element from the progeny retrovirus particles. The diseased cell may, for example, be a melanoma cell or a solid tumor cell, and the undesirable phenotype may be one selected from the group consisting of:

i) expression of a cell-surface protein associated with metastasis;

ii) expression of an mRNA encoding a cell-surface protein associated with metastasis;

iii) cell-to-cell adhesion among the melanoma cells;

iv) invasiveness of the melanoma cells;

v) survival of the melanoma cells;

vi) growth of the melanoma cells; and

vii) proliferation of the melanoma cells in a three dimensional growth environment such as the body of a mammal.

The cell-surface protein associated with metastasis may, for example, be selected from the group consisting of Mel-CAM and integrin (beta)3. The undesirable phenotype may also be angiogenesis, particularly when the diseased cell is a solid tumor cell.

The invention still further relates to a genetic suppressor element which exhibits an anti-melanoma effect. This genetic suppressor element is a polynucleotide having a length of at least about 10 nucleotide residues and is derived from at least about 10 consecutive nucleotide residues of a portion of the cDNA corresponding to Mel-CAM. The portion is selected from the group consisting of SEQ ID NOs: 1-9.

The genetic suppressor element may be complementary to or homologous with the portion of the cDNA. For example, the genetic suppressor element can have a nucleotide sequence selected from the group consisting of

a) nucleotide sequences complementary to a portion of the cDNA corresponding to Mel-CAM selected from the group consisting of SEQ ID NOs: 1-4 and 6; and

b) nucleotide sequences homologous with a portion of the cDNA corresponding to Mel-CAM selected from the group consisting of SEQ ID NOs: 5 and 7-9.

The invention yet further relates to a genetic suppressor element which exhibits an anti-angiogenesis effect in a solid tumor. This genetic suppressor element is a polynucleotide having a length of at least about 10 nucleotide residues and is derived from at least about 10 consecutive nucleotide residues of a known genetic suppressor element which inhibits expression of integrin (beta)3. The known genetic suppressor element is derived from a portion of the cDNA corresponding to integrin (beta)3 and having a nucleotide sequence derived selected from the group consisting of SEQ ID NOs: 10-21. The genetic suppressor element may be complementary to or homologous with the portion of the cDNA. For example, the known genetic suppressor element may have a nucleotide sequence selected from the group consisting of

a) nucleotide sequences complementary to a portion of the cDNA corresponding to integrin (beta)3 selected from the group consisting of SEQ ID NOs: 12, 15, and 17-19; and

b) nucleotide sequences homologous with a portion of the cDNA corresponding to integrin (beta)3 selected from the group consisting of SEQ ID NOs: 10, 11, 13, 14, 16, 20, and 21.

In another aspect, the invention relates to a method of inhibiting an undesirable phenotype of a human melanoma cell. This method comprises providing a genetic suppressor element to the cell. The genetic suppressor element is selected from the group consisting of

a) a polynucleotide having a length of at least about 10 nucleotide residues and having a nucleotide sequence complementary to at least about 10 consecutive nucleotide residues of a portion of the cDNA corresponding to Mel-CAM, wherein the portion is selected from the group consisting of SEQ ID NOs: 1-4 and 6;

b) a polynucleotide having a length of at least about 10 nucleotide residues and having a nucleotide sequence homologous with at least about 10 consecutive nucleotide residues of a portion of the cDNA corresponding to Mel-CAM, wherein the portion is selected from the group consisting of SEQ ID NOs: 5 and 7;

c) a polynucleotide having a length of at least about 10 nucleotide residues and having a nucleotide sequence complementary to at least about 10 consecutive nucleotide residues of a portion of the cDNA corresponding to integrin (beta)3, wherein the portion is selected from the group consisting of SEQ ID NOs: 12 and 15; and

d) a polynucleotide having a length of at least about 10 nucleotide residues and having a nucleotide sequence homologous with at least about 10 consecutive nucleotide residues of a portion of the cDNA corresponding to integrin (beta)3, wherein the portion is selected from the group consisting of SEQ ID NOs: 10, 11, 13, 14 and 16.

In yet another aspect, the invention relates to a method of inhibiting an undesirable phenotype of a human solid tumor cell. This method comprising providing a genetic suppressor element to the cell. The genetic suppressor element is a polynucleotide having a length of at least about 10 nucleotide residues and is derived from at least about 10 consecutive nucleotide residues of a known genetic suppressor element which inhibits expression of integrin (beta)3. The known genetic suppressor element has a nucleotide sequence derived from a portion of the cDNA corresponding to integrin (beta)3 having a nucleotide sequence selected from the group consisting of SEQ ID NOs: 10-16.

In multiple embodiments, the invention includes the method wherein a human melanoma cell is located in the body of a mammal.

In one embodiment, the invention includes a method of inhibiting an undesirable phenotype of a human solid tumor cell, the method comprising providing a genetic suppressor element to the cell, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16.

In another embodiment, the invention includes a method of treating a human having a solid tumor, which tumor exhibits an undesirable phenotype, the method comprising administering to the human a composition comprising a genetic suppressor element, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16, thereby treating the human having the solid tumor. The this method, the undesirable phenotype is selected from the group consisting of:

i) expression of a cell-surface protein associated with metastasis;

ii) expression of an mRNA encoding a cell-surface protein associated with metastasis;

iii) cell-to-cell adhesion among the melanoma cells;

iv) invasiveness of the melanoma cells;

v) survival of the melanoma cells;

vi) growth of the melanoma cells; and

vii) proliferation of the melanoma cells, wherein the melanoma cells are located in the body of a mammal.

In one aspect, this method also includes a solid tumor which is an early stage solid tumor.

In another aspect, the composition further comprises a pharmaceutically acceptable carrier.

In one embodiment, the invention encompasses a method of inhibiting the recurrence of a solid tumor, which solid tumor exhibits an undesirable phenotype, the method comprising providing a composition comprising a genetic suppressor element to the solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16; thereby inhibiting solid tumor recurrence.

In another embodiment, the invention encompasses a method of prolonging remission of a solid tumor, the method comprising providing a composition comprising a genetic suppressor element to the solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16; thereby prolonging remission of a solid tumor.

According to this method, the composition further comprises a pharmaceutically acceptable carrier, and in some embodiments, the remission of the solid tumor constitutes the absence of one or more solid tumor characteristics selected from the group consisting of metastasis, invasiveness, accelerated growth, and accelerated proliferation.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1, comprising FIGS. 1A-1G, is a series of graphs and images depicting a typical characterization of down-regulating Mel-CAM GSE's. FIGS. 1A-1E depict cell surface profiles obtained using fluorescence-activated cell sorting (FACS) analysis. The progressive enrichment of 1205Lu cells with cells expressing reduced Mel-CAM on the cell surface is shown. FACS analyses were performed using A32 monoclonal antibody against Mel-CAM as described in Example 1 herein. FIG. 1A is a cell surface expression profile of the control antibody P3X only. FIG. 1B (corresponding to L22) is a cell surface expression profile of 1205Lu cells as positive control (i.e. exhibiting normal levels of Mel-CAM expression). FIGS. 1C-1E (corresponding to L23, L24, and L25, respectively) are each a cell surface expression profile of a selected GSE clones expressing reduced amounts of Mel CAM (i.e. exhibiting normal levels of Mel-CAM expression). FIG. 1F is an image of a northern analysis depicting Mel-CAM mRNA expression in 1205Lu cells and selected GSE clones. FIG. 1G is an image of a western blot depicting Mel-CAM protein expression in 1205Lu and selected GSE clones.

FIG. 2 is a FACS analysis using Mel-CAM specific antibody, A32. Peaks A (P3X/control) and B (A32/control) represent cells which are negative and positive controls, respectively. Peak C (A32/Test/L4) represents GSE clone L4 cells, which expresses high levels of Mel-CAM.

FIG. 3, comprising FIGS. 3A, 3B, 3C, and 3D, is a quartet of nucleotide sequences. FIG. 3A is the nucleotide sequence (SEQ ID NO: 1) of the portion of the cDNA corresponding to Mel-CAM to which the GSE of clone L22 is complementary. FIG. 3B is the nucleotide sequence (SEQ ID NO: 2) of the portion of the cDNA corresponding to Mel-CAM to which the GSE of clone L23 is complementary. FIG. 3C is the nucleotide sequence (SEQ ID NO: 3) of the portion of the cDNA corresponding to Mel-CAM to which the GSE of clone L24 is complementary. FIG. 3D is the nucleotide sequence (SEQ ID NO: 4) of the portion of the cDNA corresponding to Mel-CAM to which the GSE of clone L25 is complementary.

FIG. 4, comprising FIGS. 4A, 4B, 4C, and 4D, is a quartet of bar graphs which indicate the percentage of two-color events in cell-to-cell binding experiments described herein. Data in FIG. 4A correspond to experiments involving the GSE of clone L22. Data in FIG. 4B correspond to experiments involving the GSE of clone L23. Data in FIG. 4C correspond to experiments involving the GSE of clone L24. Data in FIG. 4D correspond to experiments involving the GSE of clone L25.

FIG. 5, comprising FIGS. 5A-5D is a series of images illustrating the functional consequences of down-regulation on Mel-CAM in melanoma, such as the effect of an antisense Mel-CAM GSE on skin reconstructs in vitro. The artificial skin reconstructs were generated as described in Example 1 herein, and contained either 1205Lu control cells, shown in FIGS. 5A and 5C, or the cell line containing Mel-CAM GSE L24, shown in FIGS. 5B and 5D. These reconstructs were analyzed after 12 days in culture for their invasiveness into the dermis (FIGS. 5A and 5B) and level of apoptosis (FIG. 5C and 5D).

FIG. 6 is a graph illustrating the control of gap junctional communication by MelCAM.

FIG. 7 is a graph which depicts the influence of Mel-CAM expression on adhesion to matrix proteins. Checked bars correspond to 1205Lu, hatched bars correspond to 1205Lu/L4, and open bars correspond to 1205Lu/L22.

FIG. 8 is a graph which depicts the influence of Mel-CAM expression SBC12 cells, which do not normally express Mel-Cam, on adhesion to matrix proteins. Checked bars correspond to SBc12, hatched bars correspond to SBc12/M18, and open bars correspond to SBc12/Ad5M18.

FIG. 9 is a graph which indicates tumor growth of 1205Lu cells in SCID mice, and illustrates the effect of Mel-CAM down regulation by antisense GSE on the tumorigenicity in severe combined immunodeficient (SCID) mice. The SCID mice were injected intradermally with 10×10⁶ cells per animal the 1205Lu control cells and Mel-CAM GSE expressing L24 cells. The tumor volume was measured weekly. Each group contained 6 animals and the experiments were repeated twice. Data represented by crosses represent tumor weight in mice injected with non-transfected 1205Lu cells, and data indicated by circles represent tumor weights in mice injected with 1205Lu cells which had been transduced with the GSE of clone L24 (Mel-CAM/MOGE) prior to injection. Bars represent standard deviation.

FIG. 10, comprising FIGS. 10A, 10B, and 10C (SEQ ID NOs: 5-7, respectively), is a trio of nucleotide sequences. FIG. 10A is the nucleotide sequence (SEQ ID NO: 5) of the portion of the cDNA corresponding to Mel-CAM with which a GSE which decreased expression of Mel-CAM is homologous. FIG. 10B is the nucleotide sequence (SEQ ID NO: 6) of the portion of the cDNA corresponding to Mel-CAM to which a GSE which decreased expression of Mel-CAM is complementary. FIG. 10C is the nucleotide sequence (SEQ ID NO: 7) of the portion of the cDNA corresponding to Mel-CAM with which a GSE which decreased expression of Mel-CAM is homologous.

FIG. 11, comprising FIGS. 11A and 11B (SEQ ID NOs: 8 and 9, respectively) is a pair of nucleotide sequences of GSE clones which induced hyper-expression of Mel-CAM. FIG. 11A is a nucleotide sequence (SEQ ID NO: 8) which is homologous to the GSE referred to herein as L4, or clone L4. This GSE represents a portion of the cDNA corresponding to Mel-CAM. FIG. 11B is the nucleotide sequence (SEQ ID NO: 9) of the portion of the cDNA corresponding to Mel-CAM with which a GSE which increased expression of Mel-CAM is homologous.

FIG. 12, comprising FIGS. 12A, 12B, 12C, 12D, 12E, 12F, and 12G (SEQ ID NOs: 10-16, respectively), is a series of nucleotide sequences of GSEs which induced decreased expression of integrin (beta)3. FIG. 12A is the nucleotide sequence (SEQ ID NO: 10) of the portion of the cDNA corresponding to integrin (beta)3 with which a GSE which decreased expression of integrin (beta)3 is homologous. FIG. 12B is the nucleotide sequence (SEQ ID NO: 11) of the portion of the cDNA corresponding to integrin (beta)3 with which a GSE which decreased expression of integrin (beta)3 is homologous. FIG. 12C is the nucleotide sequence (SEQ ID NO: 12) of the portion of the cDNA corresponding to integrin (beta)3 to which a GSE which decreased expression of integrin (beta)3 is complementary. FIG. 12D is the nucleotide sequence (SEQ ID NO: 13) of the portion of the cDNA corresponding to integrin (beta)3 with which a GSE which decreased expression of integrin (beta)3 is homologous. FIG. 12E is the nucleotide sequence (SEQ ID NO: 14) of the portion of the cDNA corresponding to integrin (beta)3. with which a GSE which decreased expression of integrin (beta)3 is homologous. FIG. 12F is the nucleotide sequence (SEQ ID NO: 15) of the portion of the cDNA corresponding to integrin (beta)3 to which a GSE which decreased expression of integrin (beta)3 is complementary. FIG. 12G is the nucleotide sequence (SEQ ID NO: 16) of the portion of the cDNA corresponding to integrin (beta)3 with which a GSE which decreased expression of integrin (beta)3 is homologous.

FIG. 13, comprising FIGS. 13A, 13B, 13C, 13D, and 13E (SEQ ID NOs: 17-21, respectively), is a series of nucleotide sequences of GSEs which induced hyper-expression of integrin (beta)3. FIG. 13A is the nucleotide sequence (SEQ ID NO: 17) of the portion of the cDNA corresponding to integrin (beta)3 to which a GSE which increased expression of integrin β3 is complementary. FIG. 13B is the nucleotide sequence (SEQ ID NO: 18) of the portion of the cDNA corresponding to integrin β3 to which a GSE which increased expression of integrin β3 is complementary. FIG. 13C is the nucleotide sequence (SEQ ID NO: 19) of the portion of the cDNA corresponding to integrin (beta)3 to which a GSE which increased expression of integrin (beta)3 is complementary. FIG. 13D is the nucleotide sequence (SEQ ID NO: 20) of the portion of the cDNA corresponding to integrin (beta)3 with which a GSE which increased expression of integrin (beta)3 is homologous. FIG. 13E is the nucleotide sequence (SEQ ID NO: 21) of the portion of the cDNA corresponding to integrin (beta)3 with which a GSE which increased expression of integrin (beta)3 is homologous.

### DETAILED DESCRIPTION OF THE INVENTION

The invention relates to an improved method of screening libraries of polynucleotides in order to discover genetic suppressor elements (GSEs) which are efficacious for inhibiting undesirable phenotypes (e.g. increase in tumor thickness, invasion, or metastasis) in cells. The improved method involves using a trans-recoverable packaging-deficient retrovirus vector to deliver a plurality of polynucleotides to a population of target cells. If the polynucleotide delivered to an individual target cell is an efficacious GSE for inhibiting the undesirable phenotype, then the target cell exhibits a detectable phenotype. Target cells exhibiting the detectable phenotype are isolated from the population, and retrovirus particles comprising a nucleic acid encoding the efficacious GSE are generated from the isolated cells by providing the product(s) necessary for recovery of retrovirus-packaging ability. It is understood that the lack of a detectable phenotype may itself be a separation criterion, if cells lacking a detectable phenotype are mixed with cells which exhibit a detectable phenotype (i.e. selection may entail selecting among various detectable phenotypes, selecting among degrees of a detectable phenotype, selecting cells which exhibit a detectable phenotype from cells which do not exhibit the phenotype, or selecting cells which do not exhibit a detectable phenotype from cells which do, for example). Efficacious GSEs are thus selected from the library of polynucleotides. Retrovirus particles so generated may, for example, be subjected to one or more additional rounds of selection or used to generate isolated GSEs in the form of isolated nucleic acids.

The invention also relates to GSEs which have been identified as being efficacious for inhibiting expression or for inducing hyper-expression of two cell surface adhesion proteins, Mel-CAM and integrin (beta)3. Mel-CAM has been given various designations in the art, including MUC18, MCAM, S-endo-1 antigen, and CD146. Integrin (beta)3 is a subunit of the protein designated (alpha)v(beta)3, which protein is sometimes referred to as the vitronectin receptor, alphaIIbbeta3. Expression of Mel-CAM and integrin (beta)3 are known to be correlated with survival and growth of invasive (i.e. metastatic) melanomas (Satyamoorthy et al., 1997, Melanoma Res. 7:535-542; Xie et al., 1997, Cancer Res. 57:2295-2303; Goudon et al., 1996, Int. J. Cancer 68:650-662; Natali et al., 1997, Cancer Res. 57:1554-1560; Hieken et al., 1996, J. Surg. Res. 63:169-173). As described herein, inhibiting expression of one or both of Mel-CAM and integrin (beta)3 in melanoma cells or in cells predisposed for melanoma induces one or more of

i) a decrease in the level of mRNA corresponding to the adhesion protein, relative to the wild type level;

ii) a decrease in the level of the adhesion protein expressed at the surface of the melanoma cells, relative to the wild type level;

iii) a decrease in the incidence of cell-to-cell adhesion among the melanoma cells, relative to the wild type incidence;

iv) decreased invasiveness of the melanoma cells, relative to wild type invasiveness;

v) an increase in the incidence of apoptosis among the melanoma cells, relative to the wild type incidence;

vi) a decrease in the in vivo survival rate of the melanoma cells, relative to the wild type in vivo survival rate; and

viii) a decrease in the rate of tumor growth in vivo following injection of the melanoma cells into an animal, relative to the rate of tumor growth in vivo following injection of wild type melanoma cells into the animal.

Others have identified GSEs which are effective for inhibiting expression of various proteins (i.e. other than Mel-CAM and integrin (beta)3) using a virus-vector-transfection and cell-sorting method. The screening methods of the present invention differ from those of the prior art in that, among other things, the methods of the invention involve use of a retrovirus vector. Use of a retrovirus vector, and particularly a trans-recoverable packaging-deficient retrovirus vector, enables determination by PCR amplification of the sequence of an efficacious GSE (as in the prior art), and also permits simplified recovery of GSEs from cells in which they have their effect.

The GSEs described herein for inhibiting expression of integrin (beta)3 are also useful for selectively inhibiting angiogenesis in solid tumor cells such as melanoma cells (i.e. but not in non-tumor cells). Angiogenesis is associated with expression of receptors, such as the vitronectin receptor, which express a protein designated αv in combination with other proteins. In solid tumor cells, the ‘other’ protein with which αv is expressed is integrin (beta)3, the proteins forming an (alpha)v(beta)3 protein which functions as a vitronectin receptor. In other cells, αv associates with other proteins, and the associated proteins also function as vitronectin receptors. Others have demonstrated inhibition of binding between vitronectin and various vitronectin receptors, the inhibition being mediated by peptides designated “RGD” peptides (Brooks et al., 1994, Cell 79:1157-1164; Brooks et al., 1994, Science 264:569-571). However, RGD peptides do not act specifically upon tumor cells, and inhibit angiogenesis of cells which normally express αv. The GSEs derived from integrin (beta)3 which are described herein do not inhibit angiogenesis of cells which express αv in the absence of integrin (beta)3 expression, and therefore specifically inhibit angiogenesis in solid tumors. The GSEs derived from integrin (beta)3 may also be used to inhibit other undesirable phenotypes in solid tumors.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

A “genetic suppressor element” (“GSE”) is a polynucleotide which, when expressed in a cell which exhibits an undesirable phenotype in the absence of the GSE, induces the cell to exhibit a less-undesirable phenotype. The GSE may, for example, induce the cell to exhibit a less-severe form of the undesirable phenotype (e.g. a reduced rate of growth in a growing tumor), or it may induce ablation of the undesirable phenotype (e.g. it may ablate metastasis of tumor cells and render them non-invasive).

A “selectable marker” is a gene or a portion thereof which, when delivered to a cell using a retrovirus vector, renders the cell differentiable from cells to which the selectable marker has not been delivered. Examples of selectable markers include, by way of example, radionuclides, proteins deposited in or on the cell membrane upon infection of the cell by the retrovirus vector, and a gene which confers a detectable phenotype on a cell when it is delivered thereto.

An “undesirable phenotype” of a cell is a phenotype of a cell which the cell exhibits in an animal afflicted with a disease or disorder (e.g. a cancer), but not in the same animal when it is not afflicted with the disease or disorder.

A “wild type” phenotype of a cell is a phenotype of a cell which the cell exhibits in an animal when it is not afflicted with a disease or disorder.

A “polynucleotide” means a single strand or parallel and anti-parallel strands of a nucleic acid. Thus, a polynucleotide may be either a single-stranded or a double-stranded nucleic acid.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

A first portion of a polynucleotide is “derived from” a second portion of the same or a different polynucleotide if the first portion is either homologous with or complementary to the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked with the coding region of a gene is able to promote transcription of the coding region.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

As used herein, a “functional” biological molecule is a biological molecule in a form in which it exhibits a property by which it is characterized. A functional gene, for example, is one which, when expressed, leads to formation of a gene product (e.g. a protein or an RNA molecule) having a characteristic activity.

A “retrovirus vector” is a composition of matter which comprises an isolated nucleic acid and one or more components of a retrovirus, and which can be used to deliver the isolated nucleic acid to the interior of a cell. Numerous retrovirus vectors are known in the art including, but not limited to, the PG1EN vector having a 5′-long terminal repeat (LTR) derived from Molony murine leukemia virus and a 3′-LTR derived from Molony murine sarcoma virus.

A “packaging-deficient” retrovirus vector is a retrovirus vector which lacks at least one component of the retrovirus necessary for generating intact progeny retrovirus particles. Examples of such components include the gag, pol, and env genes and the proteins encoded thereby.

A “trans-recoverable” packaging-deficient retrovirus vector is a packaging-deficient retrovirus vector which can be induced to package its progeny retrovirus particles if one or more components of the retrovirus from which the vector is derived (e.g. the gag, pol, and env genes and the proteins encoded thereby) is provided to a cell infected with the vector.

A library of vectors “collectively comprises” a variety of elements if each of the elements is present in at least one vector of the library.

A “diseased cell” is a cell of a subject afflicted with a disease (e.g. melanoma), wherein the diseased cell has an altered phenotype, relative to the same cell in a subject not afflicted with the disease.

Description

The invention relates to compositions and methods for generating a genetic suppressor element (GSE) which suppresses an undesirable phenotype in a cell. The composition comprises a trans-recoverable packaging-deficient retrovirus vector which comprises a polynucleotide which is potentially the GSE. The method comprises screening cells which express the undesirable phenotype and which have been infected with the vector of the invention for reduction or ablation of the undesirable phenotype and then recovering progeny retrovirus particles from cells which exhibit reduction or ablation of the phenotype.

The Compositions of the Invention

The invention includes a trans-recoverable packaging-deficient retrovirus vector. This vector comprises a retrovirus having a genome which comprises a portion derived from the sequence of a cDNA corresponding to a protein expressed in a diseased cell. The genome of the vector lacks a functional copy of a gene necessary for packaging of progeny of the vector.

The region of the cDNA from which the portion is derived has a length less than about 4,000 to 7,000 nucleotide residues, more preferably less than about 1,500 to 3,000 nucleotide residues, and is preferably in the range from about 300 to about 600 nucleotide residues. Of course, the region may have a length of as little as 100, 50, 30, 20, or even 10 nucleotide residues. The region of the cDNA from which the portion is derived may be a coding region, a non-coding region, or the region may span coding and non-coding regions. The portion may be either complementary to of homologous with the region of the cDNA.

The portion is preferably operably associated with an ATG codon at the 5′-end thereof, so that the portion may be translated. The portion is preferably operably associated with one or more stop codons (e.g. one in each reading frame) at the 3′-end thereof, in order to limit translation of the portion to the sequence of the portion. As described below, an internal ribosome entry site (IRES) may be located downstream (i.e. 3′- relative to) the portion, so that a selectable marker may be translated from the same nucleic acid as the portion.

In one embodiment of the vector of the invention, the cDNA corresponds to a surface adhesion protein of the diseased cell. By way of example, the protein may be a surface adhesion protein of a melanoma cell, such as Mel-CAM or integrin (beta)3. Because expression of both of these proteins is closely linked with the metastatic state of a melanoma cell, thickness of a melanoma, and invasiveness of cells of a melanoma, the vector of the invention may be used to identify GSEs which are effective to inhibit one or more of metastasis, thickening, and invasiveness of melanoma cells in a human patient or in a sample obtained from or derived from a human patient or from another mammal. The vector of the invention may, in this embodiment, comprise a portion derived from the cDNA of Mel-CAM (e.g. derived from the cDNA listed in GenBank accession no. M28882; Lehmann et al. 1989, Proc. Natl. Acad. Sci. USA 86:9891-9895; Sers et al., 1993, Proc. Natl. Acad. Sci. USA 90:8514-8518) or a portion derived from the cDNA of integrin (beta)3 (e.g. derived from the cDNA listed in GenBank accession no. M35999; Frachet et al., 1990, Mol. Biol. Rep. 14:27-33).

An important feature of the vector of the invention is that it lacks a functional copy of a gene which is necessary for packaging of progeny of the retrovirus vector. Examples of such gene which are known in retroviruses include, for example, the gag gene, the pol gene, and the env gene. Of course, this gene may be substantially any gene which prevents packaging of progeny virus when the gene is inactivated or deleted, not merely those which are presently known.

The retrovirus vector of the invention may be substantially any retrovirus vector (i.e. a virus vector derived from any of the retroviridae). Thus, the vector may be derived from one of the Oncovirinae, one of the Spumavirinae, or one of the Lentivirinae. For example, the retrovirus vector may be a PG1EN vector, as described in Example 1 herein.

The retrovirus vector of the invention preferably comprises a selectable marker, so that cells which have been infected using the retrovirus vector may be differentiated and, if desired, separated, from cells which have not been so infected. The selectable marker may, for example, be a gene which enables an infected cell to catalyze a reaction which is not catalyzed by a non-infected cell. For example, the selectable marker may be an npt gene encoding neomycin phosphotransferase (EC 2.7.1.95). The enzyme encoded by this gene catalyzes phosphorylation of aminoglycoside antibiotics such as neomycin. Cells which infected with a retrovirus comprising a functional npt gene are able to survive exposure to G418 (a neomycin derivative known in the art as a selective agent useful in conjunction with an npt gene) or HAT (hypoxanthine, aminopterin, and thymidine) at concentrations of these compounds at which non-infected cells (i.e. cells which do not comprise a functional npt gene) are killed. In a preferable arrangement, the genome of the retrovirus vector comprises the portion, the selectable marker, and an IRES interposed therebetween. More preferably, the IRES is located 3′- relative to the portion, and the selectable marker is located 3′- relative to the IRES. In this arrangement, the selectable marker is translated from the same RNA molecule as the portion. Thus, the selectable marker indicates not only which cells have been infected, but also which cells are translating the genome of the retrovirus vector. IRESs are described, for example, by Morgan et al., 1992, Nucl. Acids Res. 20:1293-1299.

The invention also encompasses a library comprising a plurality of the vectors of the invention. At least two of the vectors collectively comprise different portions derived from the sequence of the same cDNA. Preferably, the vectors of the library collectively comprise many (i.e. at least 10, but preferably 100, 1,000, 10,000, 100,000, or more) different portions derived from the sequence of at least (and, in some embodiments, only) one cDNA. Such a library permits one to analyze the efficacy of GSEs derived from many portions of the cDNA in a single screening technique. Of course, once efficacious GSEs have been identified, as described herein, derivatives of the efficacious GSEs may be prepared. Such derivatives may comprise shortened versions of the efficacious GSEs, and may be tested, as described herein, to determine whether they are more efficacious than the previously identified GSEs. For example, as described herein, the GSEs complementary to cDNA sequences having SEQ ID NOs: 1-4 have been determined to be efficacious for inhibiting Mel-CAM expression in melanoma cells. These GSEs have lengths of 308-523 nucleotide residues (base pairs, in double-stranded form). Because it may be preferable to use GSEs having shorter lengths as pharmaceutical agents in certain circumstances, these GSEs may be derivatized to produce shorter polynucleotides, and these shorter polynucleotides may be tested using the methods described herein in Example 1, for example, to determine the efficacy of these shorter polynucleotides as GSEs for inhibiting Mel-CAM expression in melanoma cells.

The portions of a cDNA which are incorporated into a vector of the invention (or into the vectors of a library of such vectors) may, for example, be generated by random cleavage of the cDNA (e.g. using an enzyme such as DNase I or a physical method such as fluid shearing), by site specific cleavage of the cDNA (e.g. using restriction endonucleases), or by amplification of sequential regions of the cDNA (e.g. using primers designed to amplify adjacent or overlapping regions of the cDNA).

The Methods of the Invention

The invention includes a method of generating a genetic suppressor element which suppresses an undesirable phenotype in a diseased cell. This method comprises

a) contacting a retrovirus library with a population of target cells; and

b) performing at least one selection cycle using the population.

The retrovirus library comprises a plurality of retrovirus vectors of the invention. Each, or at least many, retrovirus particles in the library, comprise a selectable marker and a fragment of an RNA which is transcribed in the diseased cell. As described above, the fragment has a length less than about 1,500 to 3,000 nucleotide residues and is operably linked with an ATG codon. The retrovirus particles lacking a component necessary for packaging of progeny retrovirus particles. The target cells must be susceptible to infection by the retrovirus particles.

The selection cycle of the method of the invention comprises selecting a fraction of the target cells which express the selectable marker and which exhibit suppression of the undesirable phenotype. For example, if the diseased cell is a melanoma cell, then the undesirable phenotype may be any one or more of the following:

i) expression of a cell-surface protein (e.g. Mel-CAM or integrin (beta)3) associated with metastasis;

ii) expression of an mRNA encoding a cell-surface protein associated with metastasis;

iii) cell-to-cell adhesion among the melanoma cells;

iv) invasiveness of the melanoma cells;

v) survival of the melanoma cells;

vi) growth of the melanoma cells; and

vii) proliferation of the melanoma cells in a three dimensional growth environment, such as within the body of a mammal. Each of these phenotypes may be detected using a variety of well known techniques including, for example, those described herein in Example 1. Of course, the method of assessing one or more of these phenotypes is not critical. Substantially any method of detecting these phenotypes may be employed in the methods of the invention. If the selectable marker used is (as in Example 1), the npt gene, then expression of the selectable marker may be detected as the ability of the cells to survive in the presence of G418 or HAT. Cells which exhibit expression of the selectable marker and suppression of the undesirable phenotype are selected. Selected cells are segregated from non-segregated cell using any method known in the art. By way of example, a cell-surface protein may be bound with a detectably (e.g. fluorescently or radiographically) labeled antibody which binds specifically with that protein, and cells which are linked with at least a selected amount of the label may be separated from cells which are linked with less label using a flow cytometer equipped with a detector capable of detecting the label.

Other methods of selecting cells are well known in the art and are included in the methods of the invention. By way of example, cells exhibiting differential expression of Mel-CAM may be separated on the basis of dye transfer following cell-to-cell adhesion in the presence of EDTA, by microdissecting non-invasive cells in artificial human skin reconstructs, by high throughput screening for secreted products of Mel-CAM effector gene expression, or by permitting apoptosis of cells in suspension cultures of Mel-CAM-transfected cells in the presence of EDTA. Further by way of example, cells exhibiting differential expression of integrin (beta)3 may be separated on the basis of adhesion to ligands of that protein (e.g. vitronectin, fibronectin, fibrinogen, VWF, and the like), by isolation of cells which are resistant to RGD peptides, by microdissecting non-invasive cells in artificial human skin reconstructs, or by high throughput screening for secreted products of integrin β3 effector gene expression. Performance of each of these methods is within the skill of the skilled artisan, given the guidance provided by this disclosure.

According to the method of the invention, it is preferred that at least two selection cycles are performed and that cells of the selected fraction are propagated between the selection cycles. Propagation of selected cells potentially improves the resolution of detection for cells which exhibit suppressed undesirable phenotypes, and permits replication of the genome (i.e. within the genome of the target cells) of the retrovirus vector prior to a subsequent round of selection. In this way, the proportion of retrovirus vectors which encode GSEs which suppress the undesirable phenotype with high efficacy may be increased, relative to the original (i.e. prescreening) proportion of that vector in the library, thereby permitting detection of efficacious GSEs.

An advantage of the methods of the invention, relative to prior art methods is that efficacious GSEs may be more easily recovered following screening by the method of the invention than by prior art methods. According to the methods of the invention, GSEs may be recovered from target cells following one or more rounds of selection by providing to the target cells the progeny-virus-packaging component which the retrovirus vector of the invention lacks. The selected target cells generate progeny retrovirus particles. If necessary or desired, the GSE(s) may be isolated from the progeny retrovirus particles using standard methods (e.g. PCR).

The identity of the target cells used in the screening method of the invention is not critical. However, it is important that the virus vector of the invention be able to infect the target cells (i.e. that the target cells are susceptible to infection therewith) and that the target cells express a detectable phenotype which is either identical to or corresponds to an undesirable phenotype. For example, as described herein, expression of the cell surface adhesion proteins designated Mel-CAM and integrin (beta)3 has been correlated with a number of phenotypes associated with metastasis of melanoma cells, including, for example, invasiveness and in vivo survival of the melanoma cells. While it is preferred that melanoma cells be used as the target cells in the screening methods of the invention, it is recognized that any cell type which is capable of expressing Mel-CAM, integrin (beta)3, or both, on its surface (and which, of course, are susceptible to infection with the virus vector of the invention) may be used as target cells for identifying GSEs which are efficacious for inhibiting a melanoma-associated phenotype. Similarly, it is preferred that a diseased cell type be used as the target cell in the screening methods of the invention. Nonetheless, any cell type which exhibits a phenotype which may be correlated with an undesirable phenotype of a diseased cell may be used as the target cell in these methods.

The invention includes a method of treating a human having a solid tumor, which tumor exhibits an undesirable phenotype, the method comprising administering to the human a composition comprising a genetic suppressor element, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16, thereby treating the human having the solid tumor. The this method, the undesirable phenotype is selected from the group consisting of:

i) expression of a cell-surface protein associated with metastasis;

ii) expression of an mRNA encoding a cell-surface protein associated with metastasis;

iii) cell-to-cell adhesion among the melanoma cells;

iv) invasiveness of the melanoma cells;

v) survival of the melanoma cells;

vi) growth of the melanoma cells; and

vii) proliferation of the melanoma cells, wherein the melanoma cells are located in the body of a mammal.

In one aspect, this method also includes a solid tumor which is an early stage solid tumor.

In another aspect, the composition further comprises a pharmaceutically acceptable carrier.

In one embodiment, the invention encompasses a method of inhibiting the recurrence of a solid tumor, which solid tumor exhibits an undesirable phenotype, the method comprising providing a composition comprising a genetic suppressor element to the solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16; thereby inhibiting solid tumor recurrence.

In another embodiment, the invention encompasses a method of prolonging remission of a solid tumor, the method comprising providing a composition comprising a genetic suppressor element to the solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16; thereby prolonging remission of a solid tumor.

According to this method, the composition further comprises a pharmaceutically acceptable carrier, and in some embodiments, the remission of the solid tumor constitutes the absence of one or more solid tumor characteristics selected from the group consisting of metastasis, invasiveness, accelerated growth, and accelerated proliferation.

The GSEs of the Invention

The invention furthermore includes individual GSEs which have identified, as described herein, as GSEs which exhibit an anti-melanoma effect when provided to melanoma cells. These GSE include, for example, GSEs complementary to a nucleotide sequence selected from the group consisting of SEQ ID NOs: 1-4, 6, 12, and 15 and GSEs homologous with a nucleotide sequence selected from the group consisting of SEQ ID NOs: 5, 7, 10, 11, 13, 14, and 16. However, it is recognized that smaller GSEs which are complementary to only a portion (i.e. at least about 10, 20, or 30) consecutive nucleotide residues of a portion of these sequences are also likely to be efficacious GSEs. Although not every GSE cDNA derived from these sequences has yet been constructed and tested, it is merely a matter of straight-forward experimentation, using the methods described herein (e.g. in Example 1) to differentiate the efficacious GSEs derived from these sequences from those having lower, or no, efficacy. The invention thus includes each of these derivatives GSEs.

The GSEs of the invention are preferably incorporated into a pharmaceutical composition. This composition may be administered to a mammal such as a human in order to inhibit an undesirable phenotype of a melanoma cell in the mammal. Such pharmaceutical compositions may be administered to a mammal diagnosed as being afflicted with melanoma, to a mammal suspected of being afflicted with melanoma, or to cells obtained from a mammal known or suspected of being afflicted with melanoma. Contacting the melanoma cells with the pharmaceutical composition of the invention suppresses one or more characteristic phenotypes of melanoma cells (e.g. invasiveness, metastasis, survival at improper body locations, uncontrolled growth, etc.).

The invention thus encompasses the preparation and use of medicaments and pharmaceutical compositions comprising a GSE as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. Administration of one of these pharmaceutical compositions to a subject is useful for inhibiting a phenotype associated with a diseased cell (e.g. a melanoma cell) in the subject, as described elsewhere in the present disclosure. The active agent of the invention may be administered in the form of the GSE alone (e.g. as a polynucleotide of DNA or another nucleic acid described herein) or contained within a virus vector. The virus vector may be a retrovirus vector, as used in the screening method of the invention, or substantially any other type of vector which may be used to deliver the GSE or a nucleic acid encoding the GSE to a diseased cell. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition and which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other mammals.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0. 1% and 100% (w/w) active ingredient.

In addition to the active ingredient, a pharmaceutical composition of the invention may further comprise one or more additional pharmaceutically active agents. Particularly contemplated additional agents include other agents known to inhibit the phenotype of the diseased cell (e.g. an anti-neoplastic agent together with a GSE for inhibiting a phenotype associated with a melanoma cell).

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intraarterial, intramuscular, or intrasternal injection and intravenous, intraarterial, or kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules, in multi-dose containers containing a preservative, or in single-use devices for auto-injection or injection by a medical practitioner. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

It is understood that the ordinarily skilled physician will readily determine and prescribe an effective amount of the compound to inhibit the undesirable phenotype in the subject. In so proceeding, the physician may, for example, prescribe a relatively low dose at first, subsequently increasing the dose until an appropriate response is obtained. It is further understood, however, that the specific dose level for any particular subject will depend upon a variety of factors including the activity of the specific compound employed, the age, body weight, general health, gender, and diet of the subject, the time of administration, the route of administration, the rate of excretion, any drug combination, and the severity or prevalence of the phenotype to be inhibited.

Another aspect of the invention relates to a kit comprising a pharmaceutical composition of the invention and an instructional material. As used herein, an “instructional material” includes a publication, a recording, a diagram, or any other medium of expression which is used to communicate the usefulness of the pharmaceutical composition of the invention for inhibiting the phenotype of a diseased cell in a subject. The instructional material may also, for example, describe an appropriate dose of the pharmaceutical composition of the invention. The instructional material of the kit of the invention may, for example, be affixed to a container which contains a pharmaceutical composition of the invention or be shipped together with a container which contains the pharmaceutical composition. Alternatively, the instructional material may be shipped separately from the container with the intention that the instructional material and the pharmaceutical composition be used cooperatively by the recipient.

The invention also includes a kit comprising a pharmaceutical composition of the invention and a delivery device for delivering the composition to a subject. By way of example, the delivery device may be a squeezable spray bottle, a metered-dose spray bottle, an aerosol spray device, an atomizer, a dry powder delivery device, a self-propelling solvent/powder-dispensing device, a syringe, a needle, a tampon, or a dosage measuring container. The kit may further comprise an instructional material as described herein.

It is not intended that the GSEs of the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule (see, e.g., Fasbender et al., 1996, J. Biol. Chem. 272:6479-6489, relating to polylysine condensation of DNA in the form of adenovirus).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach, IRL Press, Oxford, England). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate, phosphorothioate, phosphorodithioate, phosphoramidate, methoxyethyl phosphoramidate, formacetal, thioformacetal, diisopropylsilyl, acetamidate, carbamate, dimethylene-sulfide (—CH₂—S—CH₂), dimethylene-sulfoxide (—CH₂—SO—CH₂), dimethylene-sulfone (—CH₂—SO₂—CH₂), 2′-O-alkyl, and 2′-deoxy-2′-fluoro phosphorothioate internucleoside linkages are well known in the art (e.g. Uhlmann et al., 1990, Chem. Rev. 90:543-584; Schneider et al., 1990, Tetrahedron Lett. 31:335).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size and type of the nucleic acid to be purified and on the characteristics of any molecules, structure, or organisms with which it may be associated. It is furthermore contemplated that the nucleic acid may comprise nucleotide residues other than the five naturally occurring bases, adenine, guanine, thymine, cytosine, and uracil.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLE 1

Antisense Mel-CAM Genetic Suppressor Elements Inhibit Invasion and Tumorigenicity of Human Melanoma Cells

In the experiments presented in this example, genetic suppressor elements (GSEs) were generated which inhibit expression of Mel-CAM, a heterophilic cell-cell adhesion molecule of the immunoglobulin superfamily. These GSEs decreased Mel-CAM expression in melanoma cells. The GSEs were generated from fragments of cDNA corresponding to Mel-CAM which were inserted into a retrovirus vector. The retrovirus vector lacked the gag, pol, and env genes necessary for assembly of progeny vectors. Cells transformed with retrovirus vector comprising a GSE exhibited depressed levels of Mel-CAM, relative to wild type cells of the same type. GSE-expressing cells were separated from wild type cells by labeling the cells with a detectable antibody which bound specifically with Mel-CAM and then sorting cells by flow cytometry.

Four DNA GSEs having lengths from 309 and 524 base pairs were identified which induced strong inhibition of Mel-CAM expression, melanoma cell aggregation, melanoma invasion in skin reconstructs from epidermis to dermis, and tumorigenicity of metastatic melanoma cells in severe combined immunodeficiency (SCID) mice. In addition, one GSE was identified which induced hyper-expression of Mel-CAM.

The materials and methods used in the experiments presented in this Example are now described.

Cell Culture and Characterization of High and Low Mel-CAM-Expressing Melanoma Cells

Metastatic melanoma cell line 1205Lu and radial growth phase- (RGP-) like melanoma cell lines, WM1552C and SBC12,were grown in MCDB153/L15 medium (Sigma Chemical Co., St. Louis, Mo.) supplemented with insulin and 2% (v/v) fetal bovine serum, as described (Satyamoorthy et al., 1997, Melanoma Res. 7(Suppl. 2):S35-S42). The amphotropic cell line designated PA317 and the ecotropic cell line designated GP+E87 were maintained in Dulbecco's modified Eagle's medium with 10% serum.

Fluorescence-activated cell sorting (FACS) analysis and cell sorting were performed using a monoclonal antibody (mAb) designated A32 in an Epics Elite™ flow analyzer (Coulter Corporation, Hialeah, Fla.) as described (Shih et al., 1994, Cancer Res. 54:2514-2520).

For Northern blot analysis, total RNA was probed using cDNAs corresponding to Mel-CAM and GAPDH, as described (Kraus et al., 1997, Melanoma Res. 7(Suppl. 2):S75-S81). Western blot analysis was performed using mAb A32 or rabbit anti-Mel-CAM polyclonal antibodies, with enhanced chemiluminescence. These analyses were performed using an antibody chemiluminescence detection kit obtained from Amersham Lifesciences (Arlington Heights, Ill.).

Aggregation of melanoma cells was assessed using a two-color assay in which two suspensions from the same cell preparation were stained using either a fluorescent green dye (5-sulfofluorescein diacetate, sodium salt {SFDA} at 50 micrograms per milliliter) or a fluorescent red dye (hydroxyethidium {HE} at 40 micrograms per milliliter) for 60 minutes prior to mixing the suspensions, as described (Degen et al., 1998, Am. J. Pathol. 152:805-813). After 30 minutes incubation at 37° C., cells were fixed in 2% (v/v) paraformaldehyde and analyzed using a flow cytometer. The results presented herein are expressed as the percentage of two-colored events (i.e. indicative of cell-to-cell adhesion), relative to the total green plus red events.

The capacity of melanoma cells to invade skin was approximated by assessing the ability of the cells to invade artificial skin reconstructs, as described (Hsu et al., 1998, Am. J. Pathol. 153:1435-1442). Briefly, human foreskin dermal fibroblasts suspended in a preparation of rat tail collagen were placed on a precast collagen gel and allowed to constrict the collagen for 6 days. Melanoma cells were then mixed with epidermal keratinocytes at a 1:5 ratio and seeded onto the surface of the dermal constructs. After 5 days, cultures were lifted to the air-liquid level to allow stratification of epidermal keratinocytes. After 10 days, the skin reconstructs were harvested, fixed in paraformaldehyde, embedded in paraffin, and sectioned and stained with hematoxylin and eosin. Apoptosis was evaluated using the ApoTag® (Oncor, Gaithersburg, Md.) in situ apoptosis detection kit, per the manufacturer's instructions.

Tumorigenicity was assessed in groups of 6 SCID mice which were injected subcutaneously with 10⁶ melanoma cells. Tumor volume in the mice was estimated weekly.

The student's t-test was used for all statistical comparisons.

Construction of a Randomly Fragmented Retrovirus Library

A randomly fragmented library of coding and non-coding sequences of cDNA corresponding to Mel-CAM was prepared, as described (Lehmann et al., 1989, Proc. Natl. Acad. Sci. USA 86:9891-9895). Approximately 100 nanograms of the cDNA fragments were subjected to random priming reaction and cloned bidirectionally into the retrovirus vector designated PG1EN, as described (Morgan et al., 1992, Nucl. Acids Res. 20:1293-1299; Pestov et al., 1994, Proc. Natl. Acad. Sci. USA 91:12549-12553), except that NotI cloning sequences were introduced into the compatible NotI site in the retrovirus vector in order to facilitate cloning. Vector PG1EN does not comprise a functional gag gene, a functional pol gene, or a functional env gene.

A plasmid library comprising about 10⁵ independent recombinant clones was used to transfected the retrovirus packaging cell lines designated PA317 (a HAT-resistant cell line) and GP+E86 using a standard calcium-phosphate precipitation method. The recombinant retroviruses were produced from the HAT- and G418 (450 micrograms per milliliter)-selected clones by “ping-pong” selection (Bunnell et al., 1996, In: Retrovirus mediated gene transfer in viral genome methods, CRC Press Inc., Boca Raton, Fla., pp. 3-23). Briefly, PA317 cells were mixed with the library and infected. The PA317 cells were then mixed with GP+E86 cells. GP+E86 cells are not HAT-resistant, and produce progeny virus vector particles at high titer. By mixing vector-infected PA317 cells with GP+E86 cells, a high percentage of PA317 cells were infected with the vector. HAT was added to the mixture to kill GP+E86 cells, and then G418 was added to kill non-infected PA317 cells. The resulting mixture comprised PA317 producer cells, substantially all of which were infected with the vector. Alternatively, 293 human embryonic kidney cells) were transfected using the retrovirus library together with plasmids comprising functional gag, pol, and env genes. These cells secreted packaged progeny virus particles. The titer of recombinant retrovirus preparations was assessed in NIH 3T3 cells prior to transfection of melanoma cells.

Isolation and Characterization of Mel-CAM GSEs

Melanoma cells which were resistant to G418 and contained stably integrated retroviruses were sorted by flow cytometry to segregate cells which expressed Mel-CAM at low levels relative to other cells. Two types of controls were used:

a) culture supernatants obtained from mock-transfected cells (i.e. cells transfected with virus vectors which did not include the GSE-npt construct), which did not give rise to any G418-resistant clones; and

b) culture supernatants obtained from cells were transfected using empty vectors (i.e. vectors containing no cDNA fragment corresponding to Mel-CAM).

About 2×10⁷ to 3×10⁷ cells were incubated with about 1 milligram of mAb A32 at 4° C. for 1 hour prior to sorting. The 1% of the cells exhibiting the lowest levels of Mel-CAM expression were harvested, propagated to about 10⁷ cells. These cells were similarly sorted twice more to extract the 1% of cells exhibiting the lowest Mel-CAM levels prior to propagation.

After the final sorting, single cell colonies were isolated and propagated. High molecular weight DNA obtained from these single cell clones (which were designated L22-L25) were isolated and subjected to PCR in order to amplify the GSEs. The primers used for PCR were complementary to the flanking sequences of the cloning site of the vector. The PCR mixture comprised 100 nanograms of genomic DNA, 300 micromolar deoxynucleotide phosphates, 100 nanograms of each PCR primer, about 5 Units of Taq polymerase, and 0.1% (w/v) gelatin. PCR mixtures were maintained at 95° C. for 5 minutes and 30 cycles were performed wherein the mixture was maintained at:

a) 94° C. for 1 minute,

b) 60° C. for 1 minute, and

c) 72° C. for 1 minute. The final cycle included an extension time of 5 minutes at 72° C. in 1×PCR buffer containing 5 Units of Taq polymerase. PCR fragments were purified by standard electrophoretic methods, and the nucleotide sequences thereof were analyzed using an automated fluorescence sequencer.

The Mel-CAM-expression-inhibiting activity of GSEs was confirmed by transfecting melanoma clones which stably expressed Mel-CAM GSEs with expression plasmid PCL-Ampho (ImGen Corp., San Diego, Calif.) using a standard calcium-phosphate precipitation technique. This plasmid encodes the gag, pol, and env genes necessary for proper packaging of the retrovirus vector. Cell culture medium was changed after 18 hours, and supernatant containing retroviruses encoding GSE were collected after 48 hours. The presence of GSE-containing retroviruses in the culture supernatants was confirmed by dot blot analysis. These retroviruses were used to infect metastatic melanoma cells. The melanoma cells were grown in the presence of G418 in order to select transfected cells. Expression of Mel-Cam by transfected cells was assessed by FACS, Northern blot, and Western blot techniques. Identification and selection protocols for high expression of Mel-CAM due to GSE's is performed using FACS sorting analysis as described for isolation of low expression inducing GSE's except, the portion of the cells collected were within the top 1% of the population.

Mel-CAM GSEs which induced hyper expression were isolated and characterized as described above, except that identification and selection protocols for hyperexpression of Mel-CAM by GSE clones was performed using FACS sorting analysis, such that the portion of the cells collected were within the top 1% of the population.

The results of the experiments presented in this Example are now described.

Isolation of Antisense Mel-CAM GSEs

A retrovirus library was generated comprising approximately 10⁵ independent clones randomly derived from the coding and non-coding regions of the cDNA corresponding to Mel-CAM cDNA. Each clone comprised a portion of the cDNA having a length of about 300 to 500 base pairs.

Three melanoma cell lines (designated 1205Lu, WM1552, and SBC12) were transfected using the retrovirus library. Cells of the parental 1205Lu cell line are highly metastatic and express high levels of Mel-CAM. WM1552 melanoma cells express Mel-CAM at low levels and have RGP-like properties (Satyamoorthy et al., 1997, Melanoma Res. 7(Suppl. 2):S35-S42). SBC12 cells do not express detectable levels of Mel-CAM.

Following retrovirus infection, neomycin-resistant cells were selected using mAb A32 and FACS in order to segregate cells which expressed low or no Mel-CAM from other cells. A total of three rounds of selection and propagation of cells were performed. Following the third round of selection for transfected 1205Lu cells, the segregated cells exhibited 50- to 100-fold lower Mel-CAM expression, relative to non-transfected parental cells. Cells from the third round of selection were spread on culture medium to generate individual colonies corresponding to individual transfected clones. Four clones which consistently expressed low levels of Mel-CAM (i.e. down-regulation clones) were selected and one clones which expressed very high levels of Mal-CAM (i.e. up-regulation clone) was also selected.

Similar results were obtained with WM1 552C cells, except that every selected clone did not express Mel-CAM at a detectable level.

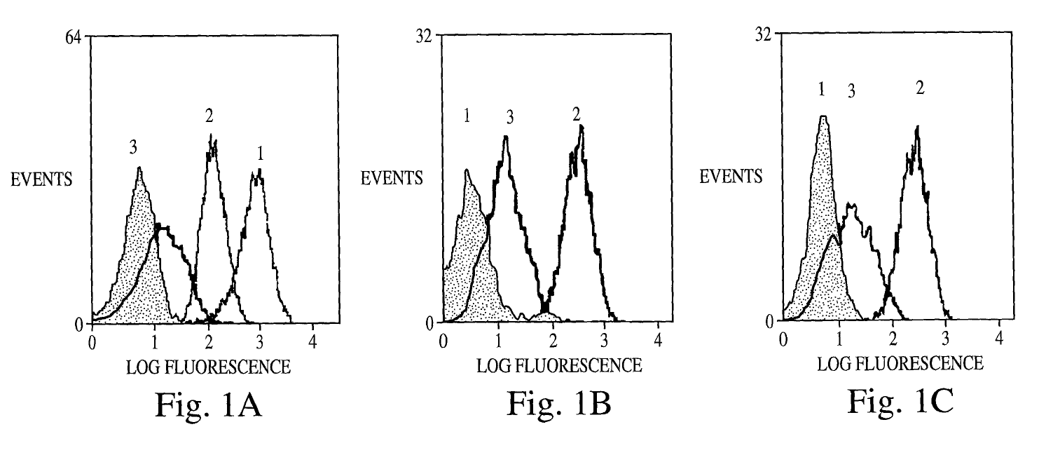

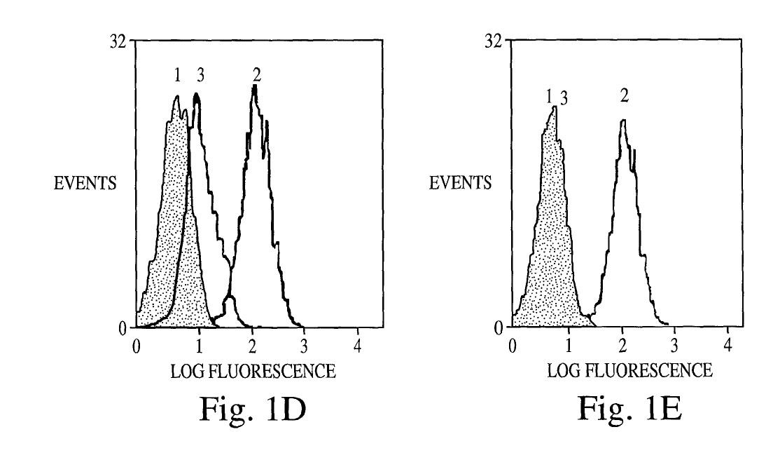

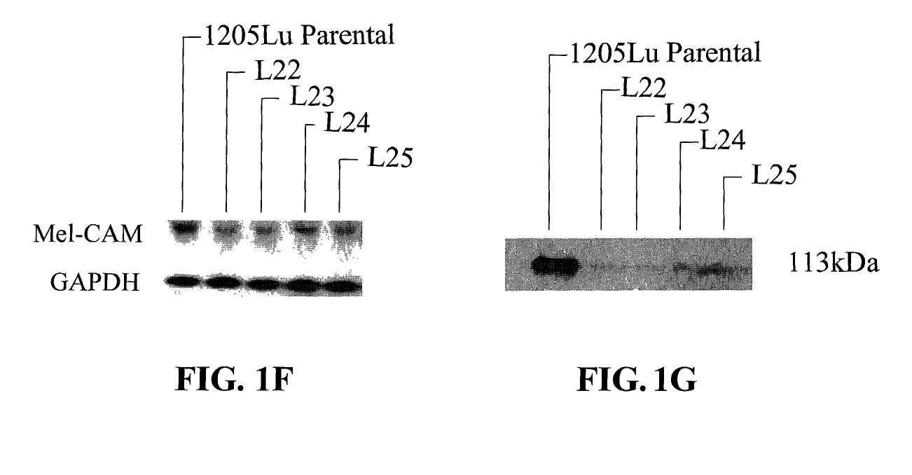

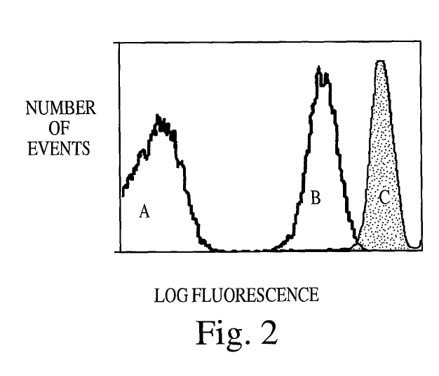

The down-regulation clones exhibited Mel-CAM mRNA levels which were 5- to 6-fold lower than wild type Mel-CAM mRNA levels. The levels of Mel-CAM protein in these clones were 5- to 9-fold lower than wild type Mel-CAM protein levels. The results of clone analysis are depicted in FIGS. 1 and 2.

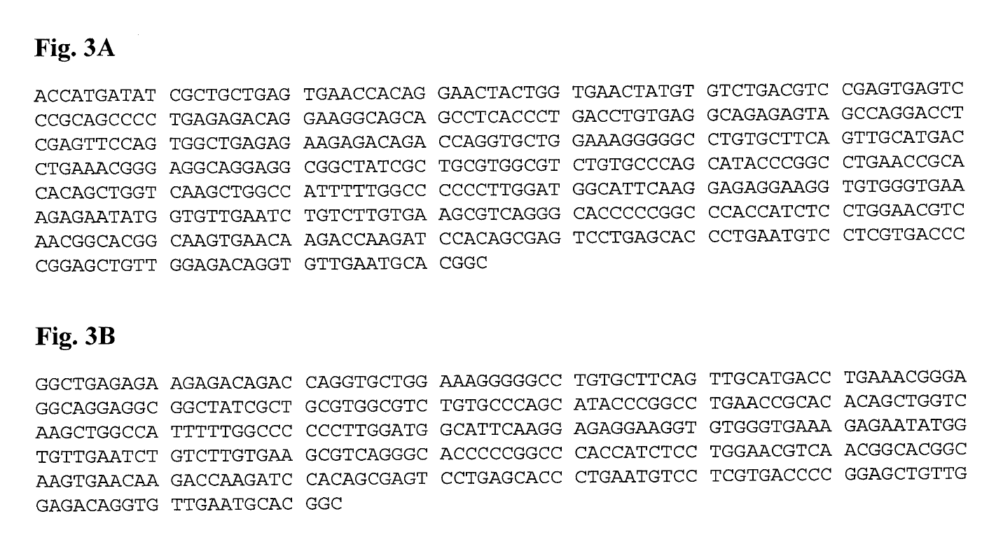

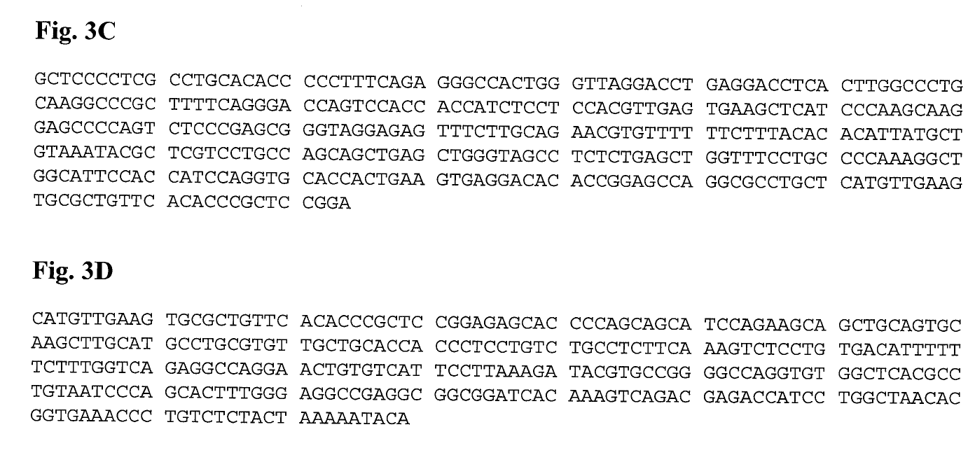

Nucleotide sequence of the GSEs isolated from the four 1205Lu-derived clones revealed that the 524-residue GSE of clone L22 spanned nucleotide residues 986 to 1509 of the cDNA corresponding to Mel-CAM, relative to the transcription start site. The 373-residue GSE of clone L23 spanned nucleotide residues 1137 to 1509 of the Mel-CAM cDNA. The 374-residue GSE of clone L24 spanned nucleotide residues 2049 to 2422 of the Mel-CAM cDNA. The 309-residue GSE of clone L25 spanned nucleotide residues 2389 to 2697 of the Mel-CAM cDNA. The GSE of clones L22 and L22 overlapped and were derived from the fourth immunoglobulin-like domain of Mel-CAM. The GSE of clone L25 was identified as spanning the C-terminal and the 3′ untranslated regions of the Mel-CAM cDNA. The nucleotide sequences (SEQ ID NOs: 1-4) of the regions of the Mel-CAM DNA to which the GSEs of clones L22-L25 are complementary are listed in FIGS. 3A-3D, respectively.

Retrovirus particles were generated from these four GSE-containing clones by transfecting cells harboring the clones with an expression plasmid containing the retroviral gag, pol, and env sequences. The virus particles thus generated were reintroduced into parental 1205Lu cells. These transfectants exhibited decreased Mel-CAM expression similar to the original isolates.

Clone L4 (SEQ ID NO:8), which was found to induce consistently high levels of Mel-CAM expression, was manipulated as described above for clones L22-L25.

Assessment of the Biological Consequences of Decreased Mel-CAM Expression in Metastatic Melanoma Cells

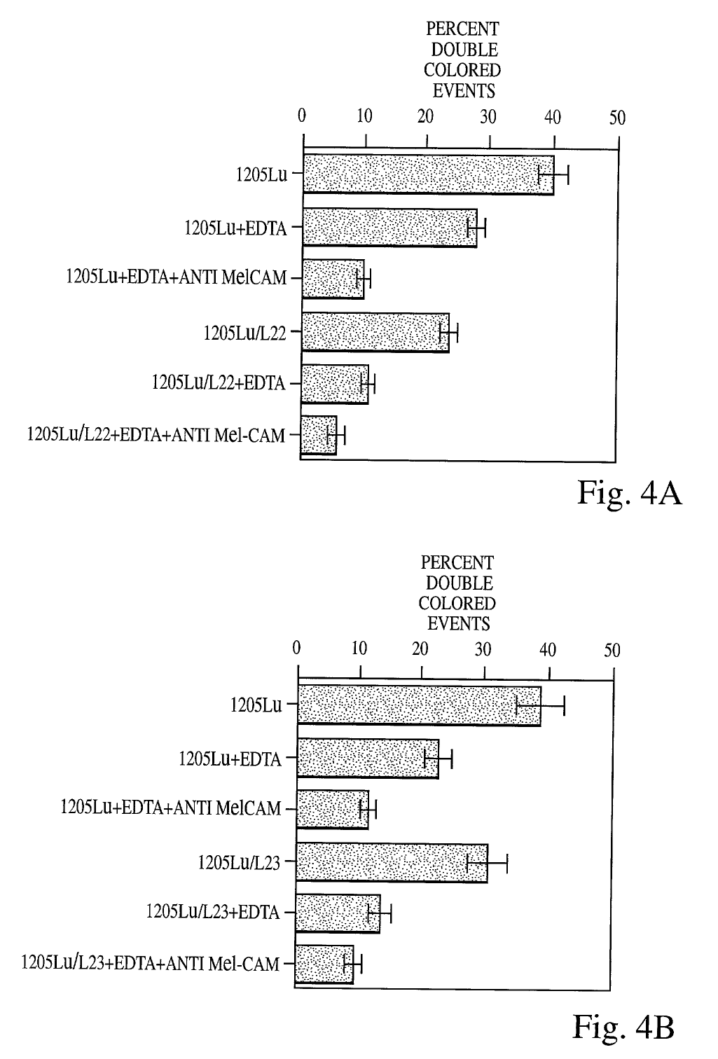

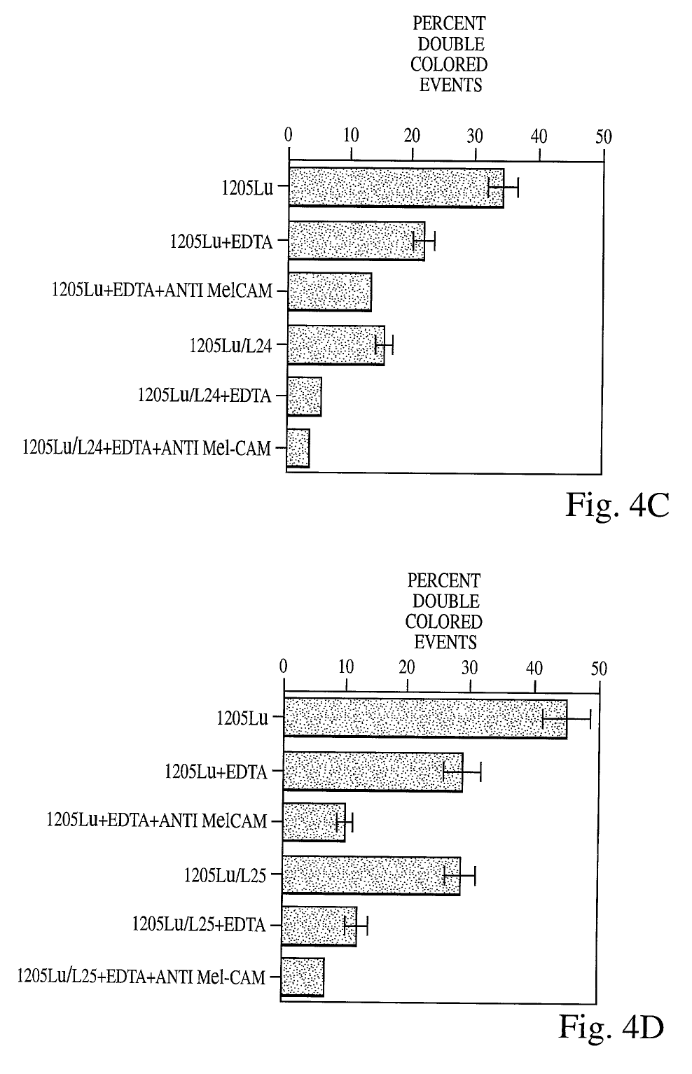

Metastatic 1205Lu melanoma cells exhibited no changes in monolayer growth patterns following Mel-CAM GSE transduction, relative to non-transduced cells. However, cell-to-cell adhesion was significantly decreased. Non-transduced 1205Lu cells aggregated readily. Cluster formation among non-transduced cells was reduced by about 40% in the presence of EDTA, which inhibits calcium-dependent binding associated with mechanisms such as those involving N-cadherin. Calcium-independent adhesion was inhibited by about 70% in non-transduced cells in the presence of a polyclonal anti-Mel-CAM antibody. In contrast, the four selected transduced 1205Lu clones which exhibited low expression of Mel-CAM, exhibited a 40 to 50% reduction in cell-to-cell adhesion, relative to wild type, non-transduced cells. In the presence of EDTA, adhesion was reduced by 70%, indicating that much of the binding which was observed in these transduced cells was calcium- or other metal-dependent binding, unlike Mel-CAM binding. Calcium-independent adhesion of the four transduced cell types decreased by only 5 to 8% in the presence of the polyclonal anti-Mel-CAM antibody, relative to adhesion in the presence of EDTA. These results of these experiments are presented in FIGS. 4A-4D.

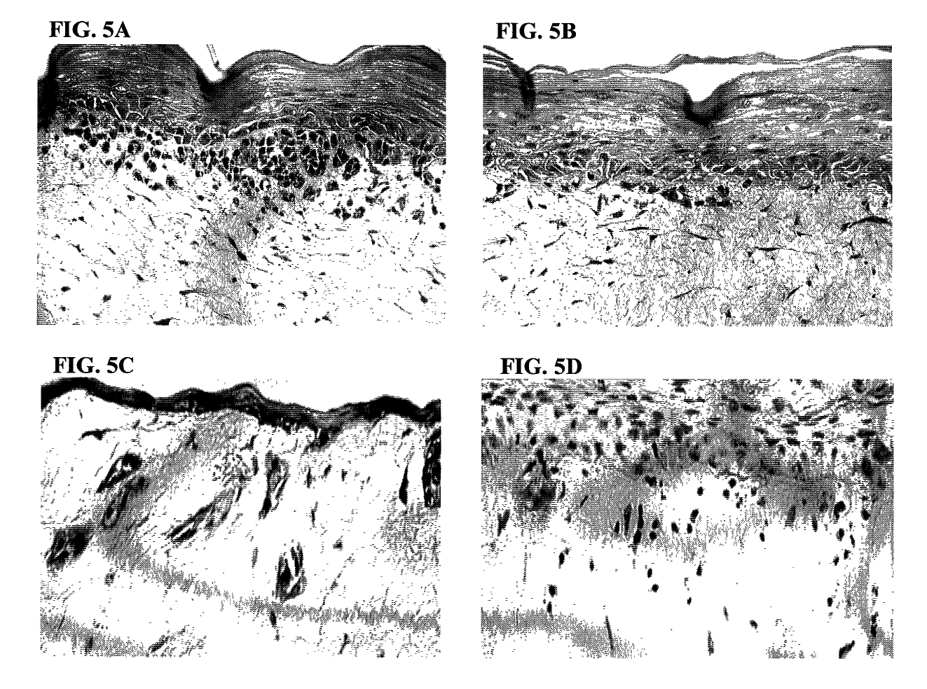

Invasiveness of 1205Lu cells with decreased Mel-CAM expression was assessed in skin reconstructs. These reconstructs comprised stratified, terminally differentiated epidermal compartments comprising keratinocytes and melanocytes and a dermal compartment comprising fibroblasts embedded in collagen gel, as described (Hsu et al., 1998, Am. J. Pathol. 153:1435-1442). When 1205Lu cells were mixed with keratinocytes prior to stratification of the epidermis, they proliferated in the basal layer of the epidermis and extensively invaded the dermal layer. In contrast, 1205Lu cells transduced with the Mel-CAM GSE of clone L22 exhibited little invasiveness; transduced cells which entered the dermal compartment exhibited signs of apoptosis such as nuclear condensation, membrane blebbing, and formation of apoptotic bodies. Transduced 1205Lu cells also were intensely stained using the ApoTag® kit according to the manufacturer's instructions. Using this kit, substantially all non-transduced 1205Lu cells appeared to be viable. The results of these experiments are depicted in FIGS. 5A-5D.

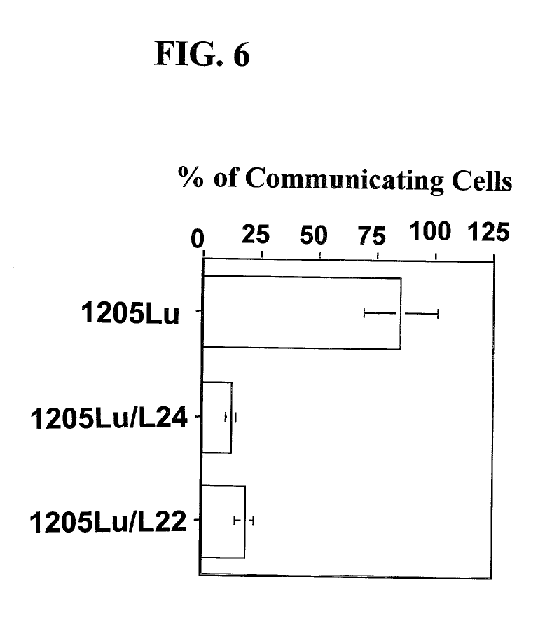

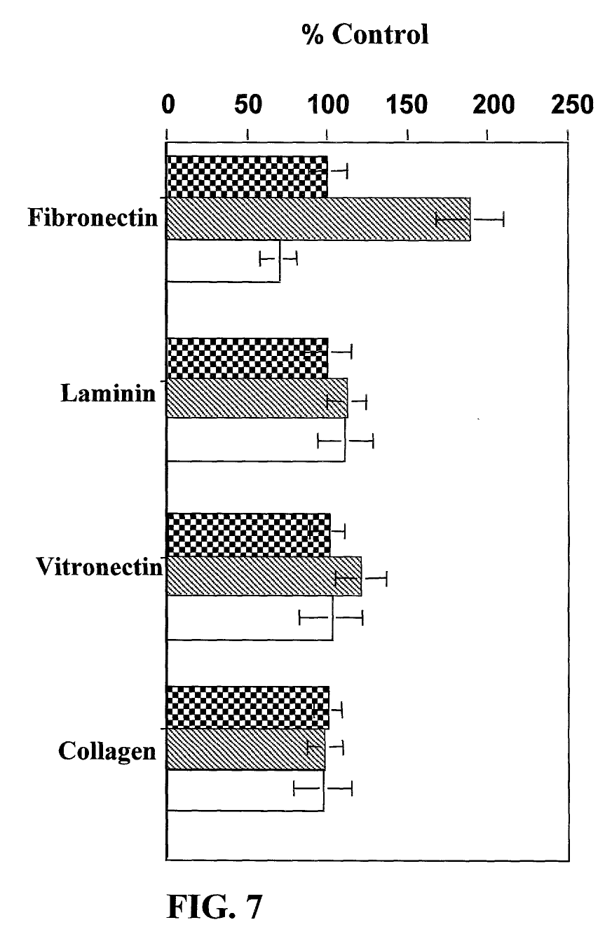

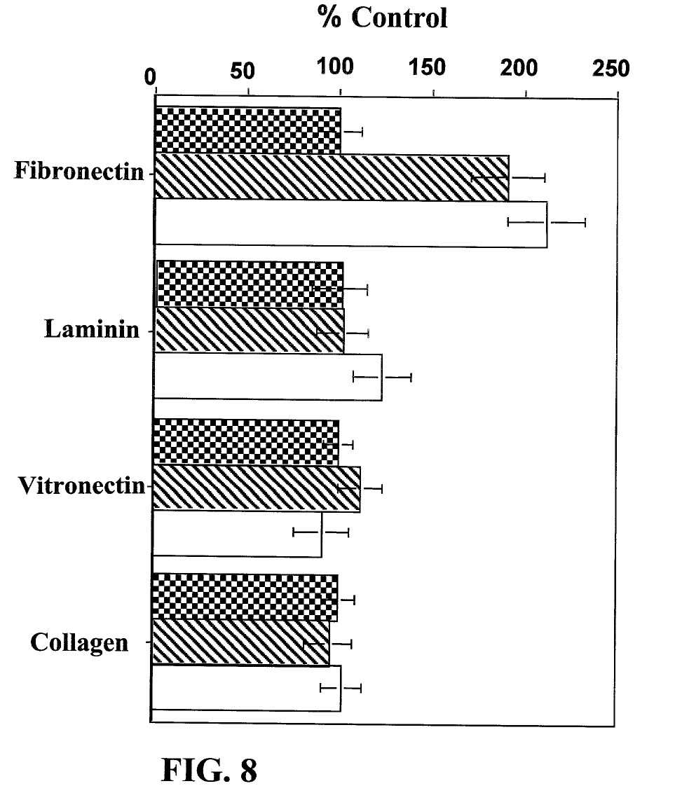

Cell-to-cell communication and adhesion were assessed using 1205Lu melanoma cells and SBC12 cells. The results of these experiments are depicted in FIGS. 6-8. As shown in FIG. 6, 1205Lu melanoma cells and clones L24 and L22 were analyzed for their ability to alter the gap junctional communication between melanoma cells. Inhibition of Mel-CAM by GSEs L24 and L22 significantly inhibited cell-cell communication between melanoma cells. As shown in FIG. 7, 1205Lu cells expressing Mel-CAM GSEs that either down regulate (cloneL22) or up-regulate (clone L4) Mel-CAM expression were evaluated for their ability to bind to various matrix proteins. High expression of Mel-CAM correlated with the enhanced ability of these cells to bind to Fibronectin but not to vitronectin, laminin and collagen. FIG. 8 depicts the results of experiments in which SBC12 cells were stably transfected with either Mel-CAM cDNA (M18) or with adenovirus expressing Mel-CAM (Ad5M18) and evaluated for their ability to bind to matrix proteins. The cells expressing Mel-CAM bound to plates coated with fibronectin but not to plates coated with vitronectin, laminin and collagen.

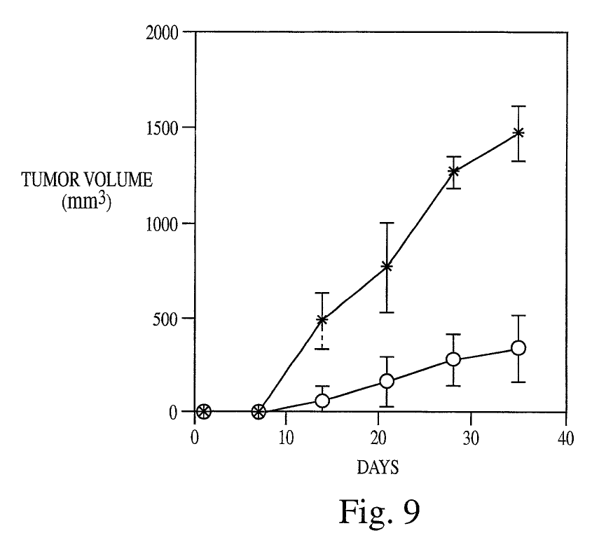

In order to assess the in vivo consequences of Mel-CAM suppression in highly tumorigenic and metastatic melanoma cells, Mel-CAM GSE-transduced 1205Lu cells (clone L22) were injected subcutaneously into SCID mice and tumor volume was monitored. GSE-transduced 1205Lu cells exhibited in vivo growth rates which were 5 times lower than the growth rates of non-transduced cells, as indicated in FIG. 9. Transduced cells were also non-metastatic, unlike the non-transduced 1205Lu cells, which exhibited consistent dissemination to the lungs of the mice.

The experiments presented in this Example demonstrate the efficacy of the methods of the invention for generating GSEs corresponding to Mel-CAM. Providing such GSEs to metastatic melanoma cells induced either down-regulation of Mel-CAM mRNA and protein expression or up-regulation of Mel-CAM mRNA and protein expression. Providing individual down-regulating GSEs to these cells resulted in:

a) loss of cell-cell aggregation, although cells maintained some cell-cell contact capability through alternative mechanisms;

b) inhibition of the cells' ability to invade the dermal compartment of a human skin reconstructs and apoptosis of the few cells which did invade this compartment; and

c) inhibition of growth of melanomas in SCID mice.

Providing individual up-regulating GSEs to these cells resulted in:

a) loss of cell-cell communication, and

b) decreased adhesion and binding of matrix proteins.

Two of the eight GSEs identified in the experiments presented in this Example were clustered within the Mel-CAM cDNA and partially overlapped at the fourth immunoglobulin-like domain. These results suggest that this domain is important for the biological functions of Mel-CAM.

Thus, the experiments presented in this Example demonstrate that the methods of the invention may be used to GSEs which modulate the biological functions of Mel-CAM, including survival, tissue invasion, and metastasis of melanoma cells.

### EXAMPLE 2

Generation of Improved GSEs for Inhibiting the Biological Functions of Mel-CAM

The GSEs described in Example 1 for inhibiting the biological functions of Mel-CAM have lengths of about 300 to 500 base pairs. It is believed that GSEs derived from the GSEs described in Example 1, and having much shorter lengths (e.g. as few as 30 to 50 base pairs) may be about equally effective for inhibiting the biological functions of Mel-CAM.

GSEs derived from those described in Example 1 may be generated by a variety of molecular biology techniques which are well known in the art. The GSEs may, for example, be randomly cleaved (e.g. physically such as by shearing or enzymatically such as by using DNAs I), specifically cleaved (e.g. using restriction endonucleases or site-specific RNA-cleaving ribozymes), amplified (e.g. using random or specifically-designed PCR primers), degraded from one or both ends (e.g. using an enzyme such as exonuclease III), or chemically synthesized (e.g. using an automated polynucleotide synthesizer).

Polynucleotides derived from the GSEs described in Example 1 may be size-fractionated to select an approximate length for the polynucleotides prior to assessing their efficacy as GSEs. Size-fractionation methods for polynucleotides are well known in the art and include, for example, gel electrophoresis, size-exclusion chromatography, and the like. The length of the polynucleotides derived from GSEs which are known to be efficacious for inhibiting biological effects of Mel-CAM may be from only slightly shorter than the known GSEs to as much as several orders of magnitude shorter. For example, when the known GSEs have lengths from about 300 to 500 base pairs, it may be sensible to derive polynucleotides having lengths from about 50 to 100 base pairs from the known GSEs, assess those polynucleotide to identify efficacious second-generation GSEs, and then derive polynucleotides having lengths from about 30 to 60 base pairs from the second-generation GSEs prior to assessing these polynucleotides for GSE-efficacy.

Polynucleotides derived from those described in Example 1 may be inserted into the retrovirus vector described in Example 1 (or in any other expression vector) and used to transduce cells which express Mel-CAM (e.g. metastatic melanoma cells such as Lu1205 cells or primary melanoma cells such as WM1552 cells). Assays for inhibition of Mel-CAM expression may be performed as described in Example 1 to identify which of the polynucleotides are efficacious GSEs for inhibiting Mel-CAM expression. Polynucleotides which exhibit a significant proportion (e.g. at least 25%, 50%, or 90% or more) of the efficacy of the GSE from which they were derived may be considered efficacious next-generation GSEs. Of course, these next-generation GSEs may be further derivated to generate shorter polynucleotides, some of which may exhibit at least most of the activity of the next-generation GSE. In this manner, GSEs having a desired efficacy (i.e. ability to inhibit one or more biological activity of Mel-CAM) and having a minimized length may be generated by the skilled artisan.

### EXAMPLE 3

Construction of GSEs for Mel-CAM and (beta)3 Integrin

The experiments presented in this Example identify the nucleotide sequences of GSEs which are efficacious for inhibiting expression of one of Mel-CAM and integrin (beta)3, and which are therefore useful for inhibiting undesirable phenotypes associated with expression of one or both of these proteins. It is known that these proteins are associated with the metastatic status of melanoma cells. In these experiments, two cell lines were used to select GSE which induced either low or excessively high expression of Mel-CAM or (beta)3 integrin. Cell line WM1552 is a RGP primary melanoma cell line and expresses low levels of Mel-CAM and (beta)3 integrin (Satyamoorthy et al., 1997, Melanoma Res. 7:535-542). Cell line 1205Lu is a highly metastatic cell line that expresses high levels of both Mel-CAM and (beta)3 integrin.

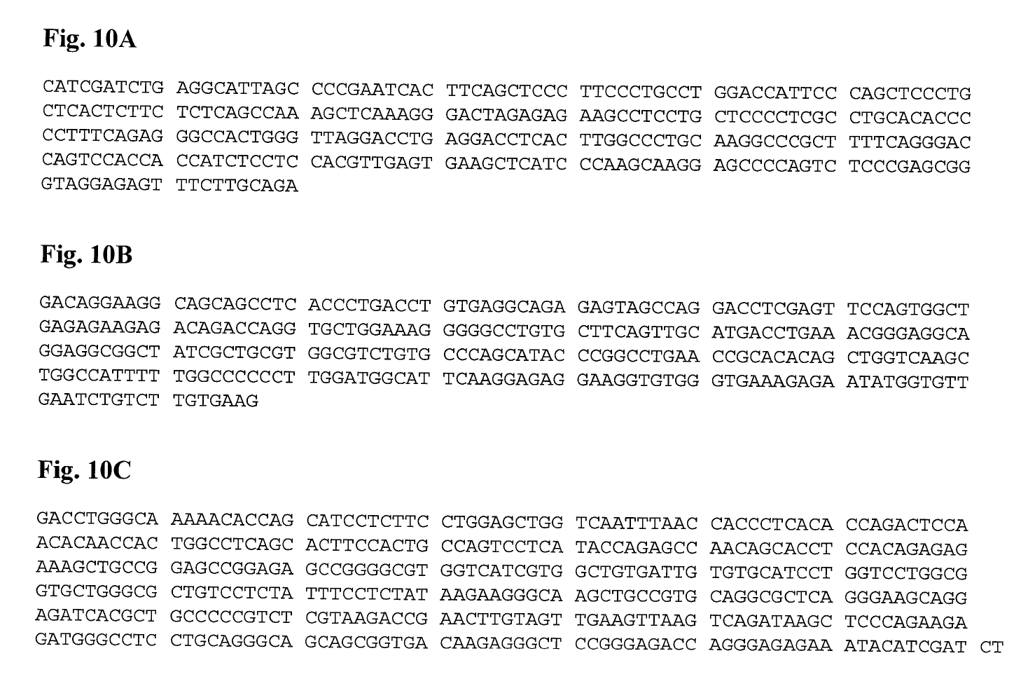

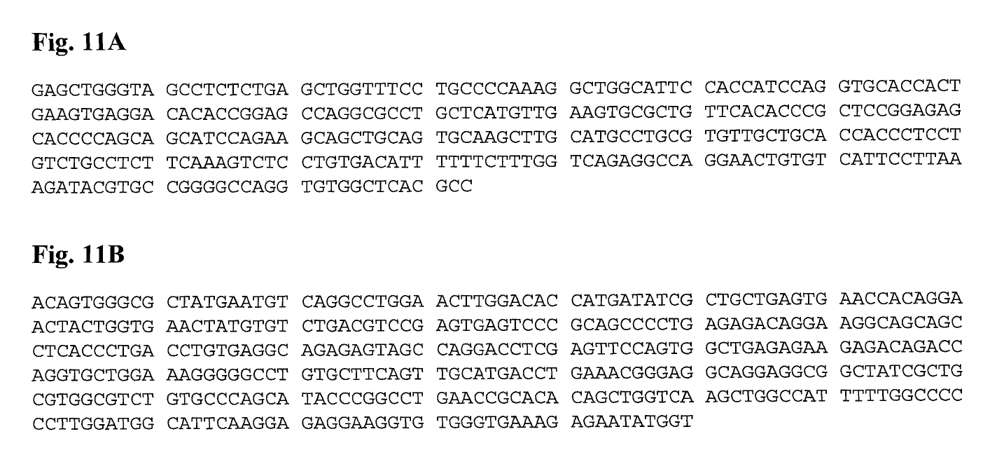

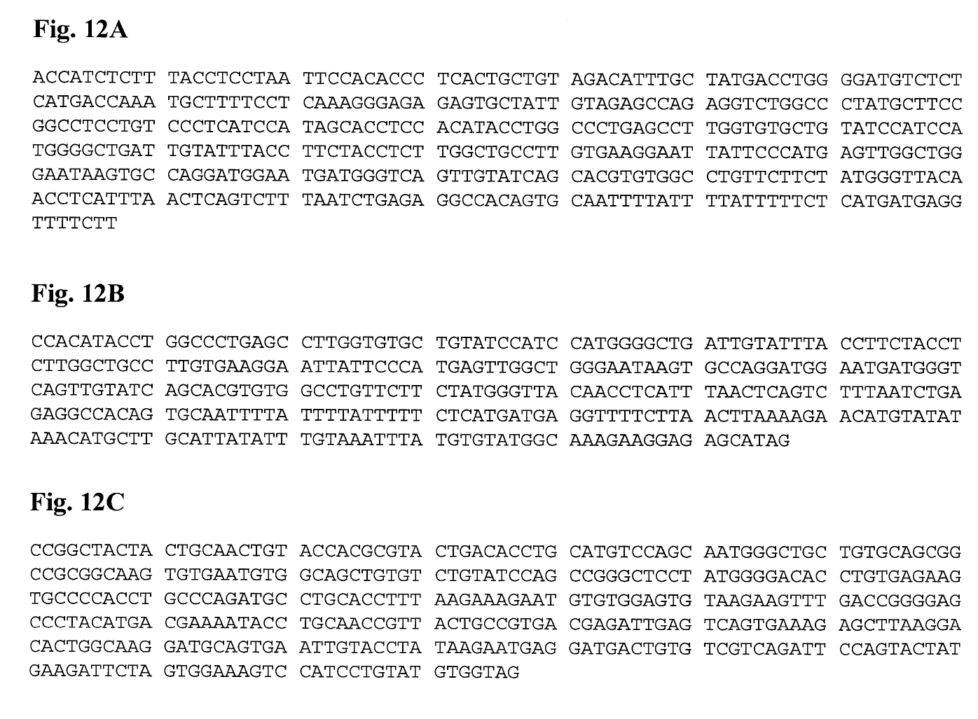

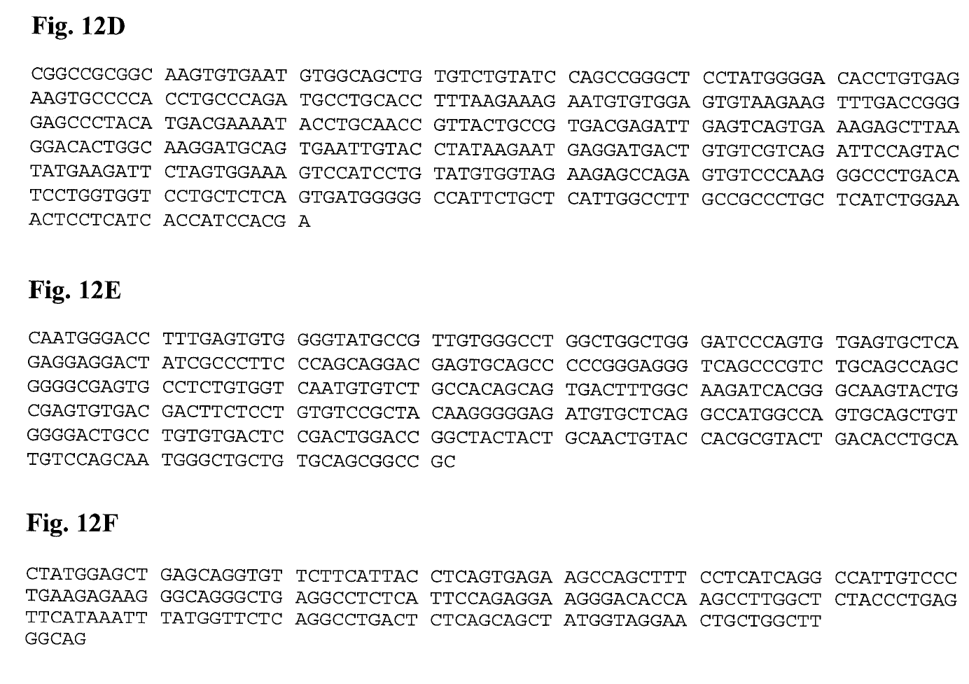

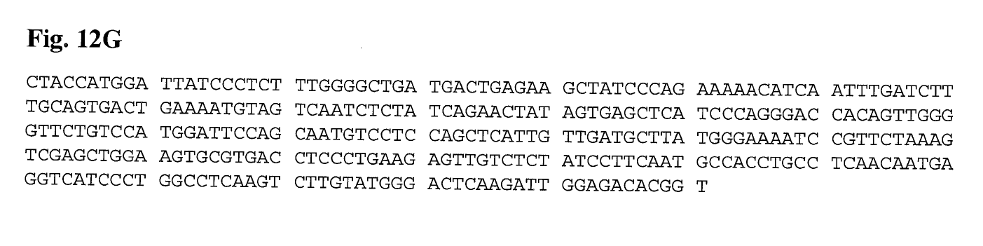

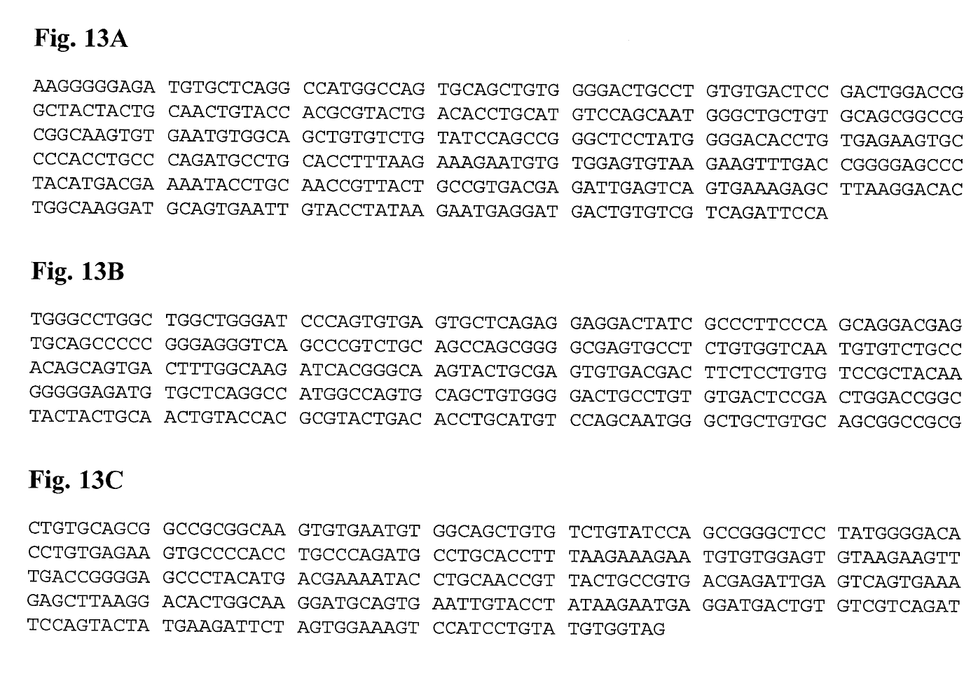

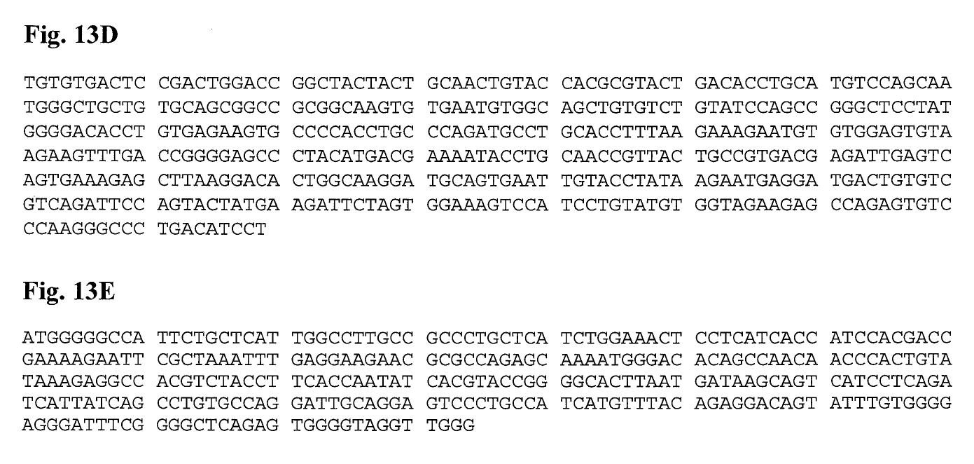

cDNAs corresponding to Mel-CAM and (beta)3 integrin were randomly cleaved as described in Example 1 to generate fragments having lengths less than about 500 base pairs. Expression of these proteins was assessed as in Example 1, using mAb A32 to detect Mel-CAM expression and mAb P3X to detect (beta)3 integrin expression. After 3 consecutive cell sorting /propagation cycles, and clones which induced at least a 10-fold increase or decrease in expression levels, relative to wild type cells of the same type were selected. DNA was isolated from each clone by amplification as described in Example 1. The sequences (SEQ ID NOs: 5-7) of portions of the cDNA corresponding to Mel-CAM to which GSE clones which induced decreased expression of Mel-CAM were complementary (SEQ ID NOs: 5 and 7) or homologous (SEQ ID NO: 6) are listed in FIGS. 10A-10C. The sequences (SEQ ID NOs: 8 and 9) of portions of the cDNA corresponding to Mel-CAM with which GSE clones which induced increased expression of Mel-CAM were homologous are listed in FIGS. 11A and 11B. FIG. 11A lists the nucleotide sequence of clone L4 (SEQ ID NO: 8). The DNA sequences (SEQ ID NOs: 10-16) of portions of the cDNA corresponding to integrin β3 to which GSE clones which induced decreased expression of integrin (beta)3 were complementary (SEQ ID NOs: 12 and 15) or homologous (SEQ ID NOs: 10, 11, 13, 14, and 16) are listed in FIGS. 12A-12G. The sequences (SEQ ID NOs: 17-21) of portions of the cDNA corresponding to integrin (beta)3 to which GSE clones which induced increased expression of integrin (beta)3 were complementary (SEQ ID NOs: 17-19) or homologous (SEQ ID NOs: 20 and 21) are listed in FIGS. 13A-13E.

Expression of integrins and CAMs other than Mel-Cam and (beta)3 integrin expressed by 1205Lu melanoma cells was not altered in cells transduced with any of these GSE clones.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A trans-recoverable packaging-deficient retrovirus vector, the vector comprising a retrovirus having a genome which comprises a portion derived from the sequence of a cDNA corresponding to a protein expressed in a diseased cell and which lacks a functional copy of a gene necessary for packaging of progeny of the vector, the portion having a length less than about 3,000 nucleotide residues.

2. The vector of claim 1, wherein the portion is complementary to the cDNA.

3. The vector of claim 1, wherein the portion is homologous with the cDNA.

4. The vector of claim 1, wherein the cDNA corresponds to a cell surface adhesion protein of the diseased cell.

5. The vector of claim 4, wherein the diseased cell is a melanoma cell.

6. The vector of claim 5, wherein the protein is selected from the group consisting of Mel-CAM and integrin (beta)3.

7. The vector of claim 1, wherein the gene is selected from the group consisting of the gag gene, the pol gene, and the env gene of the retrovirus.

8. The vector of claim 1, wherein the retrovirus vector is derived from a retrovirus selected from the group consisting of a Molony murine leukemia virus and a Molony murine sarcoma virus.

9. The vector of claim 8, wherein the retrovirus vector is a PG1EN vector comprising the portion.

10. The vector of claim 1, wherein the vector further comprises a selectable marker.

11. The vector of claim 1, wherein the portion is operably linked with a promoter/enhancer region.

12. The vector of claim 1 1, wherein the portion is operably linked with an ATG codon.

13. The vector of claim 12, wherein the portion is operably linked with a stop codon.

14. The vector of claim 13, wherein the portion is operably linked with an internal ribosome entry site and a selectable marker, the internal ribosome entry site being interposed between the portion and the selectable marker.

15. A library comprising a plurality of the vector of claim 1, wherein at least two of the vectors collectively comprise different portions derived from the sequence of the same cDNA.

16. The library of claim 15, wherein the vectors collectively comprise at least 10 different portions derived from the sequence of the cDNA.

17. The library of claim 15, wherein the portions are generated by random cleavage of the cDNA.

18. The library of claim 15, wherein the portions are generated by amplification of sequential regions of the cDNA.

19. The library of claim 15, wherein the cDNA corresponds to Mel-CAM and wherein the portions are derived from at least one region selected from the group consisting of SEQ ID NOs: 1-9.

20. The library of claim 15, wherein the cDNA corresponds to integrin (beta)3 and wherein the portions are derived from at least one region selected from the group consisting of SEQ ID NOs: 10-21.

21. A pharmaceutical composition comprising the vector of claim 1 and a pharmaceutically acceptable carrier.

22. A method of generating a genetic suppressor element which suppresses an undesirable phenotype in a diseased cell, the method comprising
a) contacting a retrovirus library with a population of target cells, the library comprising a plurality of retrovirus particles, wherein individual retrovirus particles comprise a selectable marker and a fragment of an RNA which is transcribed in the diseased cell, the fragment having a length less than about 3,000 nucleotide residues and being operably linked with an ATG codon, the retrovirus particles lacking a component necessary for packaging of progeny retrovirus particles, and the target cells being susceptible to infection by the retrovirus particles; and
b) performing at least one selection cycle using the population, the selection cycle comprising selecting a fraction of the target cells which express the selectable marker and which exhibit suppression of the undesirable phenotype.

23. The method of claim 22, wherein at least two selection cycles are performed and wherein cells of the fraction are propagated between the selection cycles.

24. The method of claim 22, further comprising providing the component to cells of the fraction, whereby progeny retrovirus particles comprising the genetic suppressor element are generated.

25. The method of claim 24, further comprising isolating the genetic suppressor element from the progeny retrovirus particles.

26. The method of claim 22, wherein the diseased cell is a melanoma cell.

27. The method of claim 26, wherein the undesirable phenotype is selected from the group consisting of:
i) expression of a cell-surface protein associated with metastasis;
ii) expression of an mRNA encoding a cell-surface protein associated with metastasis;
iii) cell-to-cell adhesion among the melanoma cells;
iv) invasiveness of the melanoma cells;
v) survival of the melanoma cells;
vi) growth of the melanoma cells; and
vii) proliferation of the melanoma cells, wherein the melanoma cells are located in the body of a mammal.

28. The method of claim 27, wherein the cell-surface protein associated with metastasis is selected from the group consisting of Mel-CAM and integrin (beta)3.

29. The method of claim 22, wherein the diseased cell is a solid tumor cell.

30. The method of claim 28, wherein the undesirable phenotype is angiogenesis.

31. The method of claim 22, wherein the diseased cell is located in the body of a mammal.

32. A genetic suppressor element which exhibits an anti-melanoma effect, the genetic suppressor element being a polynucleotide having a length of at least about 10 nucleotide residues and being derived from at least about 10 consecutive nucleotide residues of a portion of the cDNA corresponding to Mel-CAM, wherein the portion is selected from the group consisting of SEQ ID NOs: 1-9.

33. The genetic suppressor element of claim 32, wherein the genetic suppressor element is complementary to the portion of the cDNA.

34. The genetic suppressor element of claim 32, wherein the genetic suppressor element is homologous with the portion of the cDNA.

35. The genetic suppressor element of claim 32, wherein the genetic suppressor element has a nucleotide sequence selected from the group consisting of
a) nucleotide sequences complementary to a portion of the cDNA corresponding to Mel-CAM selected from the group consisting of SEQ ID NOs: 1-4 and 6; and
b) nucleotide sequences homologous with a portion of the cDNA corresponding to Mel-CAM selected from the group consisting of SEQ ID NOs: 5 and 7-9.

36. A pharmaceutical composition comprising the genetic suppressor element of claim 32 and a pharmaceutically acceptable carrier.

37. A genetic suppressor element which exhibits an anti-angiogenesis effect in a solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element being derived from a portion of the cDNA corresponding to integrin (beta)3 and having a nucleotide sequence selected from the group consisting of SEQ ID NOs: 10-21.

38. The genetic suppressor element of claim 37, wherein the genetic suppressor element is complementary to the portion of the cDNA.

39. The genetic suppressor element of claim 37, wherein the genetic suppressor element is homologous with the portion of the cDNA.

40. The genetic suppressor element of claim 37, wherein the known genetic suppressor element has a nucleotide sequence selected from the group consisting of
a) nucleotide sequences complementary to a portion of the cDNA corresponding to integrin (beta)3 selected from the group consisting of SEQ ID NOs: 12, 15, and 17-19; and
b) nucleotide sequences homologous with a portion of the cDNA corresponding to integrin (beta)3 selected from the group consisting of SEQ ID NOs: 10, 11, 13, 14, 16, 20, and 21.

41. A pharmaceutical composition comprising the genetic suppressor element of claim 37 and a pharmaceutically acceptable carrier.

42. A method of inhibiting an undesirable phenotype of a human melanoma cell, the method comprising providing a genetic suppressor element to the cell, wherein the genetic suppressor element is selected from the group consisting of
a) a polynucleotide having a length of at least about 10 nucleotide residues and having a nucleotide sequence complementary to at least about 10 consecutive nucleotide residues of a portion of the cDNA corresponding to Mel-CAM, wherein the portion is selected from the group consisting of SEQ ID NOs: 1-4 and 6;
b) a polynucleotide having a length of at least about 10 nucleotide residues and having a nucleotide sequence homologous with at least about 10 consecutive nucleotide residues of a portion of the cDNA corresponding to Mel-CAM, wherein the portion is selected from the group consisting of SEQ ID NOs: 5 and 7;
c) a polynucleotide having a length of at least about 10 nucleotide residues and having a nucleotide sequence complementary to at least about 10 consecutive nucleotide residues of a portion of the cDNA corresponding to integrin (beta)3, wherein the portion is selected from the group consisting of SEQ ID NOs: 12 and 15; and
d) a polynucleotide having a length of at least about 10 nucleotide residues and having a nucleotide sequence homologous with at least about 10 consecutive nucleotide residues of a portion of the cDNA corresponding to integrin (beta)3, wherein the portion is selected from the group consisting of SEQ ID NOs: 10, 11, 13, 14 and 16.

43. The method of claim 42, wherein the human melanoma cell is located in the body of a mammal.

44. The method of claim 42, wherein the undesirable phenotype is selected from the group consisting of:
i) expression of a cell-surface protein associated with metastasis;
ii) expression of an mRNA encoding a cell-surface protein associated with metastasis;
iii) cell-to-cell adhesion among the melanoma cells;
iv) invasiveness of the melanoma cells;
v) survival of the melanoma cells;
vi) growth of the melanoma cells; and
vii) proliferation of the melanoma cells, wherein the melanoma cells are located in the body of a mammal.

45. A method of inhibiting an undesirable phenotype of a human solid tumor cell, the method comprising providing a genetic suppressor element to the cell, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16.

46. The method of claim 45, wherein the undesirable phenotype is selected from the group consisting of:
i) expression of a cell-surface protein associated with metastasis;
ii) expression of an mRNA encoding a cell-surface protein associated with metastasis;
iii) cell-to-cell adhesion among the melanoma cells;
iv) invasiveness of the melanoma cells;
v) survival of the melanoma cells;
vi) growth of the melanoma cells; and
vii) proliferation of the melanoma cells, wherein the melanoma cells are located in the body of a mammal.

47. A method of treating a human having a solid tumor, which tumor exhibits an undesirable phenotype, the method comprising administering to the human a composition comprising a genetic suppressor element, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16, thereby treating the human having the solid tumor.

48. The method of claim 47, wherein the undesirable phenotype is selected from the group consisting of:
i) expression of a cell-surface protein associated with metastasis;
ii) expression of an mRNA encoding a cell-surface protein associated with metastasis;
iii) cell-to-cell adhesion among the melanoma cells;
iv) invasiveness of the melanoma cells;
v) survival of the melanoma cells;
vi) growth of the melanoma cells; and
vii) proliferation of the melanoma cells, wherein the melanoma cells are located in the body of a mammal.

49. The method of claim 47, wherein the solid tumor is an early stage solid tumor.

50. The method of claim 47, wherein the composition further comprises a pharmaceutically acceptable carrier.

51. A method of inhibiting solid tumor recurrence, which solid tumor exhibits an undesirable phenotype, the method comprising providing a composition comprising a genetic suppressor element to the solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16; thereby inhibiting solid tumor recurrence.

52. A method of prolonging remission of a solid tumor, the method comprising providing a composition comprising a genetic suppressor element to the solid tumor, the genetic suppressor element being a polynucleotide having a length of at least about 10 nucleotide residues and being derived from at least about 10 consecutive nucleotide residues of a known genetic suppressor element which inhibits expression of integrin (beta)3, the known genetic suppressor element having a nucleotide sequence derived from a portion of the cDNA corresponding to integrin (beta)3 having a nucleotide sequence selected from the group consisting of SEQ ID NOs: 10-16; thereby prolonging remission of a solid tumor.

53. The method of claim 52, wherein the composition further comprises a pharmaceutically acceptable carrier.

54. The method of claim 52, wherein the remission of the solid tumor constitutes the absence of one or more solid tumor characteristics selected from the group consisting of metastasis, invasiveness, accelerated growth, and accelerated proliferation.


SEQUENCE LISTING (21 sequences)

SEQ ID NO: 1 - DNA, 524 bp, Artificial Sequence
  Description of Artificial Sequence Portion of Mel-CAM cDNA to which the GSE of clone L22 is complementary
accatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacagaccaggtgctggaaagggggcctgtgcttcagttgcatgacctgaaacgggaggcaggaggcggctatcgctgcgtggcgtctgtgcccagcatacccggcctgaaccgcacacagctggtcaagctggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggc

SEQ ID NO: 2 - DNA, 373 bp, Artificial Sequence
  Description of Artificial Sequence Portion of Mel-CAM cDNA to which the GSE of clone L23 is complementary
ggctgagagaagagacagaccaggtgctggaaagggggcctgtgcttcagttgcatgacctgaaacgggaggcaggaggcggctatcgctgcgtggcgtctgtgcccagcatacccggcctgaaccgcacacagctggtcaagctggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggc

SEQ ID NO: 3 - DNA, 374 bp, Artificial Sequence
  Description of Artificial Sequence Portion of Mel-CAM cDNA to which the GSE of clone L24 is complementary
gctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatgctgtaaatacgctcgtcctgccagcagctgagctgggtagcctctctgagctggtttcctgccccaaaggctggcattccaccatccaggtgcaccactgaagtgaggacacaccggagccaggcgcctgctcatgttgaagtgcgctgttcacacccgctccgga

SEQ ID NO: 4 - DNA, 309 bp, Artificial Sequence
  Description of Artificial Sequence Portion of Mel-CAM cDNA to which the GSE of clone L25 is complementary
catgttgaagtgcgctgttcacacccgctccggagagcaccccagcagcatccagaagcagctgcagtgcaagcttgcatgcctgcgtgttgctgcaccaccctcctgtctgcctcttcaaagtctcctgtgacattttttctttggtcagaggccaggaactgtgtcattccttaaagatacgtgccggggccaggtgtggctcacgcctgtaatcccagcactttgggaggccgaggcggcggatcacaaagtcagacgagaccatcctggctaacacggtgaaaccctgtctctactaaaaataca

SEQ ID NO: 5 - DNA, 300 bp, Artificial Sequence
  Description of Artificial Sequence The portion of the cDNA corresponding to Mel-CAM with which a GSE which decreased expression of Mel-CAM is homologous.
catcgatctgaggcattagccccgaatcacttcagctcccttccctgcctggaccattcccagctccctgctcactcttctctcagccaaagctcaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcaga

SEQ ID NO: 6 - DNA, 297 bp, Artificial Sequence
  Description of Artificial Sequence The portion of the cDNA corresponding to Mel-CAM with which a GSE which decreased expression of Mel-CAM is homologous.
gacaggaaggcagcagcctcaccctgacctgtgaggcagagagtagccaggacctcgagttccagtggctgagagaagagacagaccaggtgctggaaagggggcctgtgcttcagttgcatgacctgaaacgggaggcaggaggcggctatcgctgcgtggcgtctgtgcccagcatacccggcctgaaccgcacacagctggtcaagctggccatttttggccccccttggatggcattcaaggagaggaaggtgtgggtgaaagagaatatggtgttgaatctgtcttgtgaag

SEQ ID NO: 7 - DNA, 422 bp, Artificial Sequence
  Description of Artificial Sequence The portion of the cDNA corresponding to Mel-CAM with which a GSE which decreased expression of Mel-CAM is homologous.
gacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatcacgctgcccccgtctcgtaagaccgaacttgtagttgaagttaagtcagataagctcccagaagagatgggcctcctgcagggcagcagcggtgacaagagggctccgggagaccagggagagaaatacatcgatct

SEQ ID NO: 8 - DNA, 313 bp, Artificial Sequence
  Description of Artificial Sequence A nucleotide sequence which is homologous to GSE L4.
gagctgggtagcctctctgagctggtttcctgccccaaaggctggcattccaccatccaggtgcaccactgaagtgaggacacaccggagccaggcgcctgctcatgttgaagtgcgctgttcacacccgctccggagagcaccccagcagcatccagaagcagctgcagtgcaagcttgcatgcctgcgtgttgctgcaccaccctcctgtctgcctcttcaaagtctcctgtgacattttttctttggtcagaggccaggaactgtgtcattccttaaagatacgtgccggggccaggtgtggctcacgcc

SEQ ID NO: 9 - DNA, 400 bp, Artificial Sequence
  Description of Artificial Sequence The portion of the cDNA corresponding to Mel-CAM with which a GSE which decreased expression of Mel-CAM is homologous.
acagtgggcgctatgaatgtcaggcctggaacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacagaccaggtgctggaaagggggcctgtgcttcagttgcatgacctgaaacgggaggcaggaggcggctatcgctgcgtggcgtctgtgcccagcatacccggcctgaaccgcacacagctggtcaagctggccatttttggccccccttggatggcattcaaggagaggaaggtgtgggtgaaagagaatatggt

SEQ ID NO: 10 - DNA, 427 bp, Artificial Sequence
  Description of Artificial Sequence The nucleotide sequence of the portion of teh cDNA corresponding to integrin (beta)3 with which a GSE which decreased expression of integrin (beta)3 is homologous.
accatctctttacctcctaattccacaccctcactgctgtagacatttgctatgacctggggatgtctctcatgaccaaatgcttttcctcaaagggagagagtgctattgtagagccagaggtctggccctatgcttccggcctcctgtccctcatccatagcacctccacatacctggccctgagccttggtgtgctgtatccatccatggggctgattgtatttaccttctacctcttggctgccttgtgaaggaattattcccatgagttggctgggaataagtgccaggatggaatgatgggtcagttgtatcagcacgtgtggcctgttcttctatgggttacaacctcatttaactcagtctttaatctgagaggccacagtgcaattttattttatttttctcatgatgaggttttctt

SEQ ID NO: 11 - DNA, 337 bp, Artificial Sequence
  Description of Artificial Sequence The nucleotide sequence of the portion of teh cDNA corresponding to integrin (beta)3 with which a GSE which decreased expression of integrin (beta)3 is homologous.
ccacatacctggccctgagccttggtgtgctgtatccatccatggggctgattgtatttaccttctacctcttggctgccttgtgaaggaattattcccatgagttggctgggaataagtgccaggatggaatgatgggtcagttgtatcagcacgtgtggcctgttcttctatgggttacaacctcatttaactcagtctttaatctgagaggccacagtgcaattttattttatttttctcatgatgaggttttcttaacttaaaagaacatgtatataaacatgcttgcattatatttgtaaatttatgtgtatggcaaagaaggagagcatag

SEQ ID NO: 12 - DNA, 387 bp, Artificial Sequence
  Description of Artificial Sequence The nucleotide sequence of the portion of teh cDNA corresponding to integrin (beta)3 with which a GSE which decreased expression of integrin (beta)3 is homologous.
ccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccggggagccctacatgacgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtag

SEQ ID NO: 13 - DNA, 441 bp, Artificial Sequence
  Description of Artificial Sequence The nucleotide sequence of the portion of teh cDNA corresponding to integrin (beta)3 with which a GSE which decreased expression of integrin (beta)3 is homologous.
cggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccggggagccctacatgacgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccctgacatcctggtggtcctgctctcagtgatgggggccattctgctcattggccttgccgccctgctcatctggaaactcctcatcaccatccacga

SEQ ID NO: 14 - DNA, 382 bp, Artificial Sequence
  Description of Artificial Sequence The nucleotide sequence of the portion of teh cDNA corresponding to integrin (beta)3 with which a GSE which decreased expression of integrin (beta)3 is homologous.
caatgggacctttgagtgtggggtatgccgttgtgggcctggctggctgggatcccagtgtgagtgctcagaggaggactatcgcccttcccagcaggacgagtgcagcccccgggagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgc

SEQ ID NO: 15 - DNA, 205 bp, Artificial Sequence
  Description of Artificial Sequence The nucleotide sequence of the portion of teh cDNA corresponding to integrin (beta)3 with which a GSE which decreased expression of integrin (beta)3 is homologous.
ctatggagctgagcaggtgttcttcattacctcagtgagaagccagctttcctcatcaggccattgtccctgaagagaagggcagggctgaggcctctcattccagaggaagggacaccaagccttggctctaccctgagttcataaatttatggttctcaggcctgactctcagcagctatggtaggaactgctggcttggcag

SEQ ID NO: 16 - DNA, 331 bp, Artificial Sequence
  Description of Artificial Sequence The nucleotide sequence of the portion of teh cDNA corresponding to integrin (beta)3 with which a GSE which decreased expression of integrin (beta)3 is homologous.
ctaccatggattatccctctttggggctgatgactgagaagctatcccagaaaaacatcaatttgatctttgcagtgactgaaaatgtagtcaatctctatcagaactatagtgagctcatcccagggaccacagttggggttctgtccatggattccagcaatgtcctccagctcattgttgatgcttatgggaaaatccgttctaaagtcgagctggaagtgcgtgacctccctgaagagttgtctctatccttcaatgccacctgcctcaacaatgaggtcatccctggcctcaagtcttgtatgggactcaagattggagacacggt

SEQ ID NO: 17 - DNA, 410 bp, Artificial Sequence
  Description of Artificial Sequence The nucleotide sequence of the portion of the cDNA corresponding to integrin (beta)3 to which a GSE which increased expression of integrin (beta)3 is complementary.
aagggggagatgtgctcaggccatggccagtgcagctgtggggactgcctgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccggggagccctacatgacgaaaatacctgcaaccgttactgccgtgacgagattgagtcagtgaaagagcttaaggacactggcaaggatgcagtgaattgtacctataagaatgaggatgactgtgtcgtcagattcca

SEQ ID NO: 18 - DNA, 350 bp, Artificial Sequence
  Description of Artificial Sequence The nucleotide sequence of the portion of the cDNA corresponding to integrin (beta)3 to which a GSE which increased expression of integrin (beta)3 is complementary.
tgggcctggctggctgggatcccagtgtgagtgctcagaggaggactatcgcccttcccagcaggacgagtgcagcccccgggagggtcagcccgtctgcagccagcggggcgagtgcctctgtggtcaatgtgtctgccacagcagtgactttggcaagatcacgggcaagtactgcgagtgtgacgacttctcctgtgtccgctacaagggggagatgtgctcaggccatggccagtgcagctgtggggactgcctgtgtgactccgactggaccggctactactgcaactgtaccacgcgtactgacacctgcatgtccagcaatgggctgctgtgcagcggccgcg

SEQ ID NO: 19 - DNA, 328 bp, Artificial Sequence
  Description of Artificial Sequence The nucleotide sequence of the portion of the cDNA corresponding to integrin (beta)3 to which a GSE which increased expression of integrin (beta)3 is complementary.
ctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccggggagccctacatgacgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtag

SEQ ID NO: 20 - DNA, 439 bp, Artificial Sequence
  Description of Artificial Sequence The nucleotide sequence of the portion of the cDNA corresponding to integrin (beta)3 to which a GSE which increased expression of integrin (beta)3 is complementary.
tgtgtgactccgactggaccggctactactgcaactgtaccacgcgtactgacacctgcatgtccagcaatgggctgctgtgcagcggccgcggcaagtgtgaatgtggcagctgtgtctgtatccagccgggctcctatggggacacctgtgagaagtgccccacctgcccagatgcctgcacctttaagaaagaatgtgtggagtgtaagaagtttgaccggggagccctacatgacgaaaatacctgcaaccgttactgccgtgacgagattgagtcagtgaaagagcttaaggacactggcaaggatgcagtgaattgtacctataagaatgaggatgactgtgtcgtcagattccagtactatgaagattctagtggaaagtccatcctgtatgtggtagaagagccagagtgtcccaagggccctgacatcct

SEQ ID NO: 21 - DNA, 314 bp, Artificial Sequence
  Description of Artificial Sequence The nucleotide sequence of the portion of the cDNA corresponding to integrin (beta)3 to which a GSE which increased expression of integrin (beta)3 is complementary.
atgggggccattctgctcattggccttgccgccctgctcatctggaaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaatgataagcagtcatcctcagatcattatcagcctgtgccaggattgcaggagtccctgccatcatgtttacagaggacagtatttgtggggagggatttcggggctcagagtggggtaggttggg